(19) United States
(12) Patent Application Publication
(10) Pub. No.: US 2015/0204884 A1
(43) Pub. Date: Jul. 23, 2015

(54) METHODS OF EVALUATING AND MAKING BIOLOGICS

(71) Applicant: MOMENTA PHARMACEUTICALS, INC., Cambridge, MA (US)

(72) Inventors: John Robblee, Concord, MA (US); Brian Edward Collins, Arlington, MA (US); Ganesh Kaundinya, Bedford, MA (US); Carlos J. Bosques, Arlington, MA (US)

(73) Assignee: MOMENTA PHARMACEUTICALS, INC., Cambridge, MA (US)

(21) Appl. No.: 14/404,884

(22) PCT Filed: May 31, 2013
(86) PCT No.: PCT/US2013/043675

§ 371 Date: Dec. 1, 2014

Related U.S. Application Data
(60) Provisional application No. 61/782,945, filed on Mar. 14, 2013.
(60) Provisional application No. 61/654,467, filed on Jun. 1, 2012.

(51) Int. Cl. G01N 33/68; C07K 16/00

(52) U.S. Cl. CPC G01N 33/6854; C07K 16/00; G01N 2440/38; C07K 2317/41

(57) ABSTRACT

Methods of making and evaluating antibody biological therapeutics (biosimilars) for adalimumab, rituximab and panitumumab are disclosed.

This application claims the benefit of U.S. Provisional Application No. 61/654,467, filed Jun. 1, 2012; and U.S. Provisional Application 61/782,945, filed Mar. 14, 2013.

### BACKGROUND

Biologic drugs are generally regarded to be substantially more complex and, thus, more difficult to replicate as generics than small molecule drugs, i.e., synthetic, organic compounds with well-defined structures. As a result, many in the industry believe that true generic biologics are not attainable.

### SUMMARY

The present disclosure provides, inter alia, compositions and methods that allow the evaluation, selection, and/or production (e.g., manufacture) of biologics, including, for example, biosimilars, including interchangeable, and compositions related thereto (e.g., pharmaceutical preparations). For example, the present disclosure provides methods whereby target proteins (e.g., biologics approved under a biologics license application (BLA)) are defined by characteristic signatures, and use of such signatures to evaluate, identify, and/or produce (e.g., manufacture) biologics that are similar or identical to a target protein. Compositions and methods herein are also useful, for example, in monitoring product changes and controlling product drift that may occur as a result of manufacturing changes. Methods disclosed herein allow for the evaluation of a biologic such as a test protein, e.g., a test glycoprotein. These methods include evaluating the similarity of the test protein with a target protein and, e.g., taking action based thereon. For example, the test protein can be evaluated to determine if it has a predetermined level of similarity with a target protein that is commercially available and approved for therapeutic use in humans. This is of particular use wherein one or more or all of the following conditions is present: the test protein is made by a different method than the target protein or the method used to make the target protein is not known to the maker of the test protein; the test protein is made by an entity having a different marketing approval than the entity that makes the target protein; or the test protein was approved in a process that relied on or referred to clinical information regarding the target protein for its approval. For example, the test protein is not approved under a biologics license application (BLA), a supplemental BLA or an equivalent thereof and the target protein is approved under a BLA, a supplemental BLA or an equivalent thereof. As another example, the test protein is not approved under the provisions of article 8(3) of the European Directive 2001/83/EC or an equivalent thereof. (Such reference to equivalents contemplates non-US or non-EP regulatory approval pathways.) Methods also provide for the generation of, or evaluation of, a predetermined plurality of target values for determinative test protein parameters for a test protein (e.g., the generation of, or evaluation of, a signature for a test protein), and/or use or application of such information to acquire a sameness/identity, or s/i, value describing the relationship (e.g., structural relationship) between the test protein and a preselected target protein. In some instances, an s/i value can be used to evaluate, identify, and/or produce (e.g., manufacture) a test protein. In some instances, an s/i value is a specification for release of a test protein. Accordingly, disclosed herein are, inter alia, methods of evaluating, identifying, and producing (e.g., manufacturing) a pharmaceutical product comprising a biologic.

In a first aspect, the disclosure features a method of manufacturing a pharmaceutical product comprising a biologic, e.g., a protein, e.g., a therapeutic antibody. The method includes:

producing a test biologic preparation, e.g., a test protein preparation, e.g., a test antibody preparation, wherein the test biologic is not approved under a biologics license application (BLA), a supplemental BLA, article 8(3) of the European Directive 2001/83/EC, or equivalents thereof;

processing the test biologic preparation as a pharmaceutical product if input values for one or a plurality (e.g., 2, 3, 4, 5, 6, 7, 8, 9, 10, 11, 12, 13, 14, 15, or more) of determinative test biologic parameters, e.g., determinative test protein parameters, meet a predetermined threshold for sameness with a predefined plurality of target values (a preselected criteria) for said determinative test biologic parameters, for a target biologic, thereby manufacturing a pharmaceutical product comprising a biologic, e.g., protein, e.g., therapeutic antibody.

In one embodiment, the processing comprises one or more of: processing into a drug product, e.g., formulating, combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on whether the predetermined threshold is met.

In one embodiment, the predetermined threshold for sameness is that the input values for the plurality of determinative test biologic parameters are indistinguishable from the corresponding predefined plurality of target values for the determinative parameters.

In one embodiment, the plurality of determinative test biologic parameters includes at least 4 determinative test biologic parameters. In one embodiment, the plurality of determinative test biologic parameters includes at least 5 determinative test biologic parameters. In one embodiment, the plurality of determinative test biologic parameters includes at least 6 determinative test biologic parameters. In one embodiment, the plurality of determinative test biologic parameters includes at least 7 determinative test biologic parameters. In one embodiment, the plurality of determinative test biologic parameters includes at least 8 determinative test biologic parameters. In one embodiment, the plurality of determinative test biologic parameters includes at least 9 determinative test biologic parameters.

In some embodiments, the predefined plurality of target values (the preselected criteria) is a release specification for release of the test biologic as a 351(k) licensed product, for example a biosimilar or interchangeable product, wherein a target value reflects the average value or range of values for the parameter (e.g., a range including the minimum and maximum values, and in some cases plus or minus a window of variability (e.g., +/−10%, +/−15%, +/−20%) to account for analytical and/or sample variability in the target) for any 2, 3, 4, 5, 6, 7, 8, 9, 10, 15, 20, 25, 30, 40, 50 or more samples, e.g., commercially available samples or batches, of the target protein.

In one embodiment, the test biologic preparation, e.g., test protein preparation, is a drug substance and, e.g., the processing comprises one or more of formulating; processing into a drug product; combining with a second component, e.g., an excipient or buffer. In one embodiment, the test biologic preparation, e.g., test protein preparation, is drug product.

In one embodiment, the target protein has an amino acid sequence (e.g., a primary amino acid sequence) that is at least 90%, 95%, 96%, 97%, 98%, 99% or 100% (identical) to the test protein amino acid sequence (e.g., 98%, 99% or identical to the test protein amino acid sequence), and the target protein is approved under a BLA, a supplemental BLA, article 8(3) of the European Directive 2001/83/EC, or equivalents thereof. In one embodiment, the target protein has an amino acid sequence (e.g., a primary amino acid sequence) that differs by no more than 1, 2, 3, 4, 5, 10, 15 or 20 amino acids to the test protein amino acid sequence (e.g., no more than 1, 2, 3 or 5 amino acids from the test protein amino acid sequence), and the target protein is approved under a BLA, a supplemental BLA, article 8(3) of the European Directive 2001/83/EC, or equivalents thereof.

In one embodiment, each of the values for the one or a plurality (e.g., 2, 3, 4, 5, 6, 7, 8, 9, 10, 11, 12, 13, 14, 15, or more) of determinative test biologic parameters, e.g., determinative test protein parameters, is indistinguishable from its corresponding target biologic, e.g., protein, value.

In one embodiment, the method comprises:

producing a test protein preparation, wherein the test protein is not approved under a biologics license application (BLA), a supplemental BLA, article 8(3) of the European Directive 2001/83/EC, or equivalents thereof; and

processing the test protein preparation as a pharmaceutical product if input values for one or a plurality of determinative test protein parameters (e.g., 2, 3, 4, 5, 6, 7, 8, 9, 10, 11, 12, 13, 14, 15, or more) are indistinguishable from a predefined plurality of target values for said determinative test protein parameters for a target protein, wherein the target protein has an amino acid sequence (e.g., a primary amino acid sequence) that is at least 98%, 99% or 100% identical to the test protein amino acid sequence, and wherein the target protein is approved under a BLA, a supplemental BLA, article 8(3) of the European Directive 2001/83/EC, or equivalents thereof, thereby manufacturing a pharmaceutical product comprising a protein.

In one embodiment, the determinative test biologic parameter, e.g., determinative test protein parameter, is indistinguishable from the value for that parameter (individually) in any 1, 2, 3, 4, 5, 6, 7, 8, 9, 10, 15, 20, 30, 40, 50 or more, commercially available samples, or batches, of the target biologic, e.g., protein. In one embodiment, the determinative test biologic parameter, e.g., determinative test protein parameter, is indistinguishable from the average value (or other measure of central tendency), or falls within the range (e.g., the minimum and maximum values +/− a range of variability such as +/−10%, +/−15%, +/−20% or more, or +/− one or two standard deviations) for the value, for any 2, 3, 4, 5, 6, 7, 8, 9, or 10, 15, 20, 30, 40, 50 or more, commercially available samples, or batches, of the target biologic, e.g., protein. In one embodiment, the method further comprises providing the average value (or other measure of central tendency) or range of values for a parameter for 2, 3, 4, 5, 6, 7, 8, 9, or 10 samples or batches of the target biologic, e.g., protein, and comparing it with the value for the determinative test biologic parameter, e.g., determinative test protein parameter, from the test biologic, e.g., protein.

In one embodiment, the value for the test biologic preparation, e.g., test protein preparation, is from one sample or batch of test biologic, e.g., protein (e.g., drug substance). In one embodiment, the value, e.g., an average value or range of values, for the test biologic, e.g., protein, is derived from 2, 3, 4, 5, 6, 7, 8, 9, or 10 samples or batches of test biologic, e.g., protein. In one such exemplary instance, such multiple samples or batches are pooled to produce drug product.

In one embodiment, the target biologic value, e.g., target protein value, can be determined from evaluation of one or more samples or batches, e.g., from any 1, 2, 3, 4, 5, 6, 7, 8, 9, or 10, commercially available samples or batches. In one embodiment, the target biologic value, e.g., target protein value, is the value for the parameter (individually) in any 1, 2, 3, 4, 5, 6, 7, 8, 9, or 10, commercially available samples, or batches, of the target biologic, e.g., protein. In one embodiment, the target biologic value, e.g., target protein value, is the average (or other measure of central tendency) value, or the range, for the parameter for any 2, 3, 4, 5, 6, 7, 8, 9, or 10, commercially available samples, or batches, of the target biologic, e.g., protein.

In one embodiment, the value is a range of values, for the test biologic, e.g., protein, and is derived from 2, 3, 4, 5, 6, 7, 8, 9, or 10 samples or batches of test biologic, e.g., protein and the target biologic value, e.g., target protein value, is a range, for the parameter for any 2, 3, 4, 5, 6, 7, 8, 9, or 10, commercially available samples, or batches, of the target biologic, e.g., protein.

In one embodiment, the value for a determinative test biologic parameter, e.g., determinative test protein parameter, is indistinguishable from, or falls within, the target biologic value, e.g., the target protein value, if the value of the determinative test biologic parameter, e.g., determinative test protein parameter, is within a release specification for that parameter for release as a 351(k) licensed product, for example a biosimilar or interchangeable product.

In one embodiment, the target biologic value, e.g., target protein value, is the range of variation for a characteristic, e.g., the distribution of a preselected glycan structure, of the determinative test biologic parameter, e.g., determinative test protein parameter, for a target biologic, e.g., protein. In one embodiment, the target biologic value, e.g., target protein value, for a parameter of the plurality is a function of the range of values for that parameter observed for multiple samples or batches of a target biologic, e.g., protein, e.g., commercially available samples or batches of a target biologic, e.g., protein. In one embodiment, the target biologic value is a numerical value such as a single number, or a range.

In one embodiment, the target biologic is a protein described herein. In one embodiment, the target protein is an antibody, e.g., a CDR-grafted antibody, a humanized antibody or a human antibody. In one embodiment, the target antibody is a marketed antibody described herein.

In one embodiment, the input values are for a plurality of determinative test protein parameters (e.g., at least 2, 3, 4, 5, 6, 7, 8, 9, 10, or 15 parameters, e.g., determinative test protein parameters), associated with, e.g., an intrinsic or extrinsic parameter of, said test protein.

In one embodiment, the test biologic is a glycoprotein, e.g., an antibody, e.g., an antibody described herein, and said plurality of parameters comprises at least 2, 3, 4, 5, 6, 7, 8, 9, 10, 11, 12, 13, 14, or 15 of the following parameters: amino acid sequence, amino acid oxidation, amino acid deamidation, IsoAsp/Asp, succinimide, pyroglutamate, glycation, glycan composition, free cysteine, disulfide linkage, C- and/or N-terminal truncation(s) (e.g., C-terminal lysine truncation), C-terminal amidation, product fragments, single chain disproportionality, and/or correlations. For example, in some embodiments, said plurality of parameters comprises any one or more: glycan(s) (e.g., one or more of HM3 glycan, HM5 glycan, HM6 glycan, HM7 glycan, HM8 glycan, HM9 glycan, Bisecting glycan A, Bisecting glycan B, C-terminal amino acid, e.g., lysine content, sialylated glycan, a G0F glycan, a G1F glycan, a G2F glycan, a G0 glycan, a G1 glycan, a G2 glycan, a hybrid glycan, and/or Gal alpha Gal), non-glycan post-translational modification(s) (e.g., one or more of pyroglutamate content, e.g., pyroglutamate at the N-terminus of the glycoprotein, e.g., at the N-terminus of a heavy and/or light chain of an antibody, succinimide content, free cysteine content, methionine sulfoxide content, glycation, and/or oxidation), disulfide formation, aggregate(s), higher order structure, functional (e.g., biological) activity (e.g., binding affinity etc.).

In one embodiment, the target biologic is selected from the products marketed as: Humira®, Avastin®; Rituxan®; Mabthera®; Campath®; Herceptin®; Xolair®; Prolia®; Vectibix®; ReoPro®; Zenapax®; Simulect®; Synagis®; Remicade®; Mylotarg®; Campath®; Raptiva®; Zevalin®; Erbitux®; Tysabri®; Lucentis®; Soliris®; Cimzia®; Ilaris®; Arzerra®; Bexxar®; Simponi®; Prolia®; Xgeva®; Actemra®; Benlysta®; Adcetris®; Yervoy®.

In one embodiment, one or more of the values of said determinative test biologic parameters, e.g., determinative test protein parameters, distinguishes a test biologic, e.g., protein, from a plurality of non-test biologics, e.g., proteins, but cannot distinguish a first non-test biologic, e.g., protein, from a second non-test biologic, e.g., protein, of said plurality of non-test biologics, e.g., proteins.

In one embodiment, the plurality of non-test biologics, e.g., proteins, comprises at least 2, 3, 4, 5, 6, 7, 8, 9, 10, or 15 non-test biologics, e.g., proteins. In one embodiment, the plurality of non-test biologics, e.g., proteins, consists of 2-15, 3-15, 3-10, 3-8, or 3-6 non-test biologics, e.g., proteins.

In one embodiment, one or more or all of the plurality of non-test proteins is an antibody, e.g., a humanized, CDR-grafted, or human antibody.

In one embodiment, one or more or all of the non-test proteins differs from the test protein by at least 1 amino acid residue (e.g., at least 1, 2, 3, 5, 10, 15, 20, 30, 40, 50, 70, 90, 100, 150, 200 or more amino acid residues). In one embodiment, one or more or all of the plurality of non-test proteins has at less than 95%, 90%, 85%, 80%, 70%, 60%, 50%, 40% or less sequence identity with said test protein.

In one embodiment, one, some, e.g., 2, 3, 4, or 5, or all of the following proteins are included in the plurality of non-test proteins: Avastin®; Mabthera®; Reditux®; Campath®; Herceptin®; and Xolair®. In one embodiment, some, e.g., 2, 3, 4, 5, 6, 7 or 8, or all of the following proteins are included in the plurality of non-test proteins: Avastin®; Mabthera®; Reditux®; Campath®; Herceptin®; Xolair®; Prolia®; and Vectibix®. In one embodiment, some, e.g., 2, 3, 4, 5, 6, 7, 8 or 9, or all of the following proteins are included in the plurality of non-test proteins: Humira®; Avastin®; Mabthera®; Reditux®; Campath®; Herceptin®; Xolair®; Prolia®; and Vectibix®.

In one embodiment, the method further comprises generating, or acquiring, a plurality of assessments by comparing the plurality of input values for the determinative test biologic parameters, e.g., determinative test protein parameters, with a predefined plurality of target biologic values, e.g., target protein values, for each of the plurality of parameters associated with the determinative test biologic parameters, e.g., determinative test protein parameters, and if each of the input values of the plurality meet a predetermined threshold for sameness with the target biologic values, e.g., target protein values, e.g., wherein a determinative entry is the same as, or falls within, the target biologic values, e.g., target protein value, subjecting the test biologic, e.g., protein, to further processing. E.g., based on the result of the comparison, the batch from which the test biologic preparation, e.g., test protein preparation, is taken can be processed, e.g., as described herein.

In a second aspect, the disclosure features a method of manufacturing a pharmaceutical product comprising a biologic, e.g., protein, the method comprising:

producing a test biologic preparation, e.g., test protein preparation, e.g., a therapeutic antibody preparation, wherein the test biologic is not approved under a biologics license application (BLA), a supplemental BLA, or an equivalent thereof;

receiving (or acquiring) an input value for one or each of a plurality of test biologic parameters in the test biologic preparation, wherein one or at least two of the plurality (e.g., 2, 3, 4, 5, 6, 7, 8, 9, 10, 11, 12, 13, 14, 15, or more) of test biologic parameters, e.g., test protein parameters, are determinative test biologic parameters, e.g., determinative test protein parameters, and

processing the test biologic preparation into a pharmaceutical product if input values for one or a plurality (e.g., 2, 3, 4, 5, 6, 7, 8, 9, 10, 11, 12, 13, 14, 15, or more) of determinative test biologic parameters, e.g., determinative test protein parameters, meet a predetermined threshold for sameness with a predefined plurality of target values (preselected criteria) for said determinative test biologic parameters, e.g., determinative test protein parameters, thereby manufacturing a pharmaceutical product comprising a biologic, e.g., protein.

In one embodiment, the predetermined threshold for sameness is that the input values for the plurality of determinative test biologic parameters are indistinguishable from the corresponding predefined plurality of target values for the determinative parameters.

In one embodiment, the plurality of determinative test biologic parameters includes at least 4 determinative test biologic parameters. In one embodiment, the plurality of determinative test biologic parameters includes at least 5 determinative test biologic parameters. In one embodiment, the plurality of determinative test biologic parameters includes at least 6 determinative test biologic parameters. In one embodiment, the plurality of determinative test biologic parameters includes at least 7 determinative test biologic parameters. In one embodiment, the plurality of determinative test biologic parameters includes at least 8 determinative test biologic parameters. In one embodiment, the plurality of determinative test biologic parameters includes at least 9 determinative test biologic parameters.

In some embodiments, the predefined plurality of target values (the preselected criteria) is a release specification for release of the test biologic as a 351(k) licensed product, for example a biosimilar or interchangeable product, wherein a target value reflects the average value or range of values for the parameter (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 25, 30, 40, 50 or more samples, e.g., commercially available samples or batches, of the target protein.

In one embodiment, the processing comprises one or more of: processing into a drug product, e.g., formulating; combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on whether the preselected relationship is met.

In one embodiment, the test biologic preparation is a drug substance and, e.g., the processing comprises one or more of formulating; processing into a drug product; combining with a second component, e.g., an excipient or buffer. In one embodiment, the test biologic preparation is drug product.

In one embodiment, the target protein has an amino acid sequence (e.g., a primary amino acid sequence) that is at least 90%, 95%, 96%, 97%, 98%, 99% or 100% identical to the test protein amino acid sequence (e.g., 98%, 99% or identical to the test protein amino acid sequence), and the target protein is approved under a BLA, a supplemental BLA, article 8(3) of the European Directive 2001/83/EC, or equivalents thereof. In one embodiment, the target protein has an amino acid sequence (e.g., a primary amino acid sequence) that differs by no more than 1, 2, 3, 4, 5, 10, 15 or 20 amino acids to the test protein amino acid sequence (e.g., no more than 1, 2, 3 or 5 amino acids from the test protein amino acid sequence), and the target protein is approved under a BLA, a supplemental BLA, article 8(3) of the European Directive 2001/83/EC, or equivalents thereof.

In one embodiment, each of the values for the one or plurality of determinative test biologic parameters, e.g., determinative test protein parameters, is indistinguishable from its corresponding target biologic value, e.g., target protein value.

In one embodiment, the method comprises:

producing a test protein preparation, wherein the test protein is not approved under a biologics license application (BLA), a supplemental BLA, article 8(3) of the European Directive 2001/83/EC, or equivalents thereof; and

processing the test protein preparation as a pharmaceutical product if input values for one or a plurality of determinative test protein parameters are indistinguishable from a predefined plurality of target values (preselected criteria) for said determinative test protein parameters for a target protein, wherein the target protein has an amino acid sequence (e.g., a primary amino acid sequence) that is at least 98%, 99% or identical to the test protein amino acid sequence, and wherein the target protein is approved under a BLA, a supplemental BLA, article 8(3) of the European Directive 2001/83/EC, or equivalents thereof, thereby manufacturing a pharmaceutical product comprising a protein.

In one embodiment, the method comprises:

producing a test protein preparation, wherein the test protein is not approved under a biologics license application (BLA), a supplemental BLA, or an equivalent thereof;

receiving (or acquiring) an input value for each of a plurality of test protein parameters in the test protein preparation, wherein at least two of the plurality of test protein parameters are determinative test protein parameters, and

processing the test protein preparation into a pharmaceutical product if the input values for each of the determinative test protein parameters are indistinguishable from a predefined plurality of target values (preselected criteria) for said determinative test protein parameters for a target protein, wherein the target protein has an amino acid sequence that is substantially the same as the test protein amino acid sequence (e.g., the target protein has an amino acid sequence that is at least 95%, 96%, 97%, 98% or more identical to the test protein amino acid sequence or which differs by less than 10, 5, 4, 3 or less amino acids from the test protein amino acid sequence), and wherein the target protein is approved under a BLA, a supplemental BLA, or an equivalent thereof, thereby manufacturing a pharmaceutical product comprising a protein.

In a third aspect, the disclosure features a method of manufacturing a pharmaceutical product comprising a protein (e.g. a therapeutic antibody), the method comprising:

producing a test protein preparation, wherein the test protein is not approved under a biologics license application (BLA), a supplemental BLA, or an equivalent thereof;

receiving (or acquiring) an input value for each of one or a plurality of test protein parameters, in the test protein preparation wherein one or at least two of the plurality of test protein parameters are determinative test protein parameters (i.e., is a function of an input value for a parameter that can distinguish the test protein from a plurality of non-test proteins);

receiving (or acquiring) a plurality of assessments made by comparing the one or plurality of input values with a predefined plurality of target values (preselected criteria) for the determinative test protein parameters, wherein the target protein has an amino acid sequence (e.g., a primary amino acid sequence) that is substantially the same as the test protein amino acid sequence (e.g., the target protein has an amino acid sequence that is at least 95%, 96%, 97%, 98%, 99% or identical to the test protein amino acid sequence or which differs by less than 10, 5, 4, 3 or less amino acids from the test protein amino acid sequence), and wherein the target protein is approved under a BLA, a supplemental BLA, or an equivalent thereof; and

processing the test protein preparation into a pharmaceutical product (e.g., a pharmaceutical composition) if input values for the plurality of determinative test protein parameters meet a predetermined threshold for sameness with the predefined plurality of target values for said determinative test protein parameters, thereby manufacturing a pharmaceutical product comprising a protein.

In one embodiment, the input values are for a plurality of determinative test protein parameters (e.g., at least 2, 3, 4, 5, 6, 7, 8, 9, 10, or 15 parameters, e.g., determinative test protein parameters), associated with, e.g., an intrinsic or extrinsic parameter of, said test protein.

In one embodiment, the predetermined threshold for sameness is that the input values for the plurality of determinative test biologic parameters are indistinguishable from the corresponding predefined plurality of target values for the determinative parameters.

In some embodiments, the predefined plurality of target values (the preselected criteria) is a release specification for release of the test biologic as a 351(k) licensed product, for example a biosimilar or interchangeable product, wherein a target value reflects the average value or range of values for the parameter (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 25, 30, 40, 50 or more samples, e.g., commercially available samples or batches, of the target protein.

In one embodiment, the plurality of determinative test biologic parameters includes at least 4 determinative test biologic parameters. In one embodiment, the plurality of determinative test biologic parameters includes at least 5 determinative test biologic parameters. In one embodiment, the plurality of determinative test biologic parameters includes at least 6 determinative test biologic parameters. In one embodiment, the plurality of determinative test biologic parameters includes at least 7 determinative test biologic parameters. In one embodiment, the plurality of determinative test biologic parameters includes at least 8 determinative test biologic parameters. In one embodiment, the plurality of determinative test biologic parameters includes at least 9 determinative test biologic parameters.

In one embodiment, the processing comprises one or more of: processing into a drug product, e.g., formulating; combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on whether the preselected relationship is met.

In one embodiment, the test protein preparation is a drug substance and, e.g., the processing comprises one or more of formulating; processing into a drug product; combining with a second component, e.g., an excipient or buffer. In one embodiment, the test protein preparation is drug product.

In one embodiment, the target protein has an amino acid sequence (e.g., a primary amino acid sequence) that is at least 90%, 95%, 96%, 97%, 98%, 99% or identical to the test protein amino acid sequence (e.g., 98%, 99% or identical to the test protein amino acid sequence), and the target protein is approved under a BLA, a supplemental BLA, article 8(3) of the European Directive 2001/83/EC, or equivalents thereof. In one embodiment, the target protein has an amino acid sequence (e.g., primary amino acid sequence) that differs by no more than 1, 2, 3, 4, 5, 10, 15 or 20 amino acids to the test protein amino acid sequence (e.g., no more than 1, 2, 3 or 5 amino acids from the test protein amino acid sequence), and the target protein is approved under a BLA, a supplemental BLA, article 8(3) of the European Directive 2001/83/EC, or equivalents thereof.

In one embodiment, each of the values for the plurality of determinative test protein parameters is indistinguishable from its corresponding target protein value.

In one embodiment, the method comprises:

producing a test protein preparation, wherein the test protein is not approved under a biologics license application (BLA), a supplemental BLA, or an equivalent thereof;

receiving (or acquiring) an input value for each of a plurality of test protein parameters in the test protein preparation wherein at least two of the plurality of test protein parameters are determinative test protein parameters (i.e., is a function of an input value for a parameter that can distinguish the test protein from a plurality of non-test proteins);

receiving (or acquiring) a plurality of assessments made by comparing the plurality of determinative test protein parameters with a predefined plurality of target values (preselected criteria) for said determinative test protein parameters for said determinative test protein parameters for a target protein, wherein the target protein has an amino acid sequence (e.g., a primary amino acid sequence) that is substantially the same as the test protein amino acid sequence (e.g., the target protein has an amino acid sequence that is at least 95%, 96%, 97%, 98% or more identical to the test protein amino acid sequence or which differs by less than 10, 5, 4, 3 or less amino acids from the test protein amino acid sequence), and wherein the target protein is approved under a BLA, a supplemental BLA, or an equivalent thereof; and

processing the test protein preparation into a pharmaceutical product if the input values for each of the determinative test protein parameters are indistinguishable from the predefined plurality of target values for said determinative test protein parameters for a target protein, thereby manufacturing a pharmaceutical product comprising a protein.

In a fourth aspect, the disclosure features a method of manufacturing a pharmaceutical product comprising a biologic, e.g., protein, the method comprising:

producing a test biologic preparation, e.g., a test protein preparation, wherein the test biologic is not approved under a biologics license application (BLA), a supplemental BLA, or an equivalent thereof;

obtaining a signature for the test biologic, e.g., protein, wherein the signature comprises a plurality, e.g., at least 2, of values for determinative test biologic parameters, e.g., determinative test protein parameters, e.g., at least 2, that distinguish the test biologic from a plurality of non-test biologics; and

processing the test biologic preparation, e.g., protein, into a pharmaceutical product if the signature for the test biologic meets a predetermined threshold for sameness with a predetermined signature (of the determinative test biologic parameters) for a target biologic (preselected criteria), thereby manufacturing a pharmaceutical product comprising a biologic, e.g., protein.

In one embodiment, the processing comprises one or more of: formulating; processing into a drug product; combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on whether the preselected relationship is met.

In one embodiment, the predetermined threshold for sameness is that the input values for the plurality of determinative test biologic parameters are indistinguishable from the corresponding predefined plurality of target values for the determinative parameters.

In some embodiments, the predefined plurality of target values (the preselected criteria) is a release specification for the parameter for release of the test biologic as a 351(k) licensed product, for example a biosimilar or interchangeable product, that reflects the average value or range of values for the parameter (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 25, 30, 40, 50 or more samples, e.g., commercially available samples or batches, of the target protein.

In one embodiment, the plurality of determinative test biologic parameters includes at least 4 determinative test biologic parameters. In one embodiment, the plurality of determinative test biologic parameters includes at least 5 determinative test biologic parameters. In one embodiment, the plurality of determinative test biologic parameters includes at least 6 determinative test biologic parameters. In one embodiment, the plurality of determinative test biologic parameters includes at least 7 determinative test biologic parameters. In one embodiment, the plurality of determinative test biologic parameters includes at least 8 determinative test biologic parameters. In one embodiment, the plurality of determinative test biologic parameters includes at least 9 determinative test biologic parameters.

In one embodiment, the test biologic preparation is a drug substance and, e.g., the processing comprises one or more of formulating; processing into a drug product; combining with a second component, e.g., an excipient or buffer. In one embodiment, the test biologic preparation is drug product.

In one embodiment, the target protein has an amino acid sequence that is at least 90%, 95%, 96%, 97%, 98%, 99% or identical to the test protein amino acid sequence (e.g., 98%, 99% or identical to the test protein amino acid sequence), and the target protein is approved under a BLA, a supplemental BLA, article 8(3) of the European Directive 2001/83/EC, or equivalents thereof. In one embodiment, the target protein has an amino acid sequence that differs by no more than 1, 2, 3, 4, 5, 10, 15 or 20 amino acids to the test protein amino acid sequence (e.g., no more than 1, 2, 3 or 5 amino acids from the test protein amino acid sequence), and the target protein is approved under a BLA, a supplemental BLA, article 8(3) of the European Directive 2001/83/EC, or equivalents thereof.

In one embodiment, the method comprises:

producing a test protein preparation, wherein the test protein is not approved under a biologics license application (BLA), a supplemental BLA, or an equivalent thereof;

obtaining a signature for the test protein, wherein the signature comprises a plurality, e.g., at least 2, of values for determinative test protein parameters (e.g., at least 2) that distinguish the test protein from a plurality of non-test proteins; and

processing the test protein preparation into a pharmaceutical product if the signature for the test protein is indistinguishable from a predetermined signature (of the determinative test protein parameters) for a target protein, wherein the target protein has an amino acid sequence that is substantially the same as the test protein amino acid sequence (e.g., the target protein has an amino acid sequence that is at least 95%, 96%, 97%, 98% or more identical to the test protein amino acid sequence or which differs by less than 10, 5, 4, 3 or less amino acids from the test protein amino acid sequence), and wherein the target protein is approved under a BLA, a supplemental BLA, or an equivalent thereof, thereby manufacturing a pharmaceutical product comprising a protein.

In a fifth aspect, the disclosure features a method of evaluating a test biologic preparation, e.g., a test protein preparation, the method comprising:

receiving (or acquiring) input values for one or a plurality of determinative test biologic parameters, e.g., determinative test protein parameters, wherein each determinative test biologic parameter is a function of an input value that can distinguish the test biologic from a plurality of non-test sample biologics; and

generating, or acquiring, a plurality of assessments by comparing the input values for the one or plurality of determinative test biologic parameters with a predefined plurality of target biologic values (preselected criteria) for each of the determinative test biologic parameters for the target biologic; and

if each of the input values of the one or the plurality of determinative test biologic parameters meet a predetermined threshold for sameness with the target biologic, e.g., wherein a determinative entry is the same as, or falls within, the target biologic value, subjecting the test biologic to further processing,

provided that the target biologic is a commercially available product, e.g., a BLA approved product, and the test sample is an unapproved product or an approved product that was approved by a secondary approval process that referred to the target biologic.

In one embodiment, the predetermined threshold for sameness is that the input values for the plurality of determinative test biologic parameters are indistinguishable from the corresponding predefined plurality of target values for the determinative parameters.

In some embodiments, the predefined plurality of target values (the preselected criteria) is a release specification for the parameter for release of the test biologic as a 351(k) licensed product, for example a biosimilar or interchangeable product, that reflects the average value or range of values for the parameter (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 25, 30, 40, 50 or more samples, e.g., commercially available samples or batches, of the target protein.

In one embodiment, the plurality of determinative test biologic parameters includes at least 4 determinative test biologic parameters. In one embodiment, the plurality of determinative test biologic parameters includes at least 5 determinative test biologic parameters. In one embodiment, the plurality of determinative test biologic parameters includes at least 6 determinative test biologic parameters. In one embodiment, the plurality of determinative test biologic parameters includes at least 7 determinative test biologic parameters. In one embodiment, the plurality of determinative test biologic parameters includes at least 8 determinative test biologic parameters. In one embodiment, the plurality of determinative test biologic parameters includes at least 9 determinative test biologic parameters.

In one embodiment, the further processing comprises one or more of: processing into a drug product, e.g., formulating; combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on whether the preselected relationship is met.

In one embodiment, the test biologic preparation is a drug substance and, e.g., the further processing comprises one or more of formulating; processing into a drug product; combining with a second component, e.g., an excipient or buffer. In one embodiment, the test biologic preparation is drug product.

In one embodiment, the target protein has an amino acid sequence that is at least 90%, 95%, 96%, 97%, 98%, 99% or identical to the test protein amino acid sequence (e.g., 98%, 99% or identical to the test protein amino acid sequence), and the target protein is approved under a BLA, a supplemental BLA, article 8(3) of the European Directive 2001/83/EC, or equivalents thereof. In one embodiment, the target protein has an amino acid sequence that differs by no more than 1, 2, 3, 4, 5, 10, 15 or 20 amino acids to the test protein amino acid sequence (e.g., no more than 1, 2, 3 or 5 amino acids from the test protein amino acid sequence), and the target protein is approved under a BLA, a supplemental BLA, article 8(3) of the European Directive 2001/83/EC, or equivalents thereof.

In one embodiment, the input value of the determinative test biologic parameter is the same as, or falls within, the target biologic value, if the determinative entry falls within the average value or range of values (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 30, 40, 50 or more, commercially available samples, or lots, of the target biologic.

In one embodiment, the method further comprises, providing the range of values for a parameter found in 2, 3, 4, 5, 6, 7, 8, 9, or 10 samples or lots of commercially available target biologic and comparing that range with the input value for the parameter from the test biologic preparation.

In one embodiment, the input value for the test biologic is a function of the value (e.g., an average or a range) for the parameter from 2, 3, 4, 5, 6, 7, 8, 9, or 10 samples or lots of test biologic.

In one embodiment, the input value of a determinative test biologic parameter is the same as, or falls within, the target biologic value, if the determinative entry is within a release specification for that parameter for release as a 351(k) licensed product, for example a biosimilar or interchangeable product.

In one embodiment, responsive to the step of generating or acquiring the plurality of assessments, the methods include generating an s/i value, wherein the determinative test biologic parameters are (selected) such that, if the generated s/i value meets a predetermined threshold for s/i, the consideration of additional determinative test biologic parameters (or non-determinative entries) does not affect whether the generated s/i value meets said threshold.

In some aspects, the disclosure provides methods of manufacturing a pharmaceutical product comprising a recombinant antibody, wherein such methods include: providing a sample of a test recombinant antibody preparation having a first amino acid sequence with at least 95%, 98%, 99%, or 100% identity to SEQ ID NO:1 and a second amino acid sequence with at least 95%, 98%, 99%, or 100% identity to SEQ ID NO:2; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:1 and second amino acid sequence with 100% identity to SEQ ID NO:1, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation includes a first amino acid sequence with 100% identity to SEQ ID NO:1 and a second amino acid sequence with 100% identity to SEQ ID NO:2. In some instances, the acquiring step includes acquiring an input value for a plurality of determinative entries, and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) include, but are not limited to: parameter number 1 shown in Table 2; parameter number 2; parameter number 3 shown in Table 2; parameter number 1 shown in Table 1 and parameter number 2 or parameter number 3 shown in Table 2. In some instances, such determinative parameter(s) can further comprise parameter number 3 shown in Table 2. In some instances, the determinative parameter(s) can include one or more, at least one (including 1, 2, 3, 4, 5, 6, 7, 8, 9, 10, 11, 12, 13, 14, 15, 16, 17, 18, 19, 20, 21, or 22), or all, of determinative parameter numbers 3, 4, 1, 2, 7, 27, 25, 13, 16, 17, 8, 19, 12, 14, 15, 20, 22, 29, 30, 31, 35, and/or 33 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (including 1, 2, 3, 4, 5, 6, 7, 8, 9, 10, 11, 12, 13, 14, 15, 16, 17, 18, 19, 20, 21, or 22), or all, of determinative parameter numbers 3, 4, 1, 2, 7, 27, 25, 13, 16, 17, 8, 19, 12, 14, 15, 20, 22, 29, 30, 31, 35, and/or 33 shown in Table 2. In some instances, the at least one input value is directly obtained using a method provided in TABLE 3. In some instances, processing the test recombinant antibody preparation into a pharmaceutical product comprising a recombinant antibody preparation includes combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein such methods include: providing a sample of a test recombinant antibody preparation having a first amino acid sequence with at least 95%, 98%, 99%, or 100% identity to SEQ ID NO:3 and a second amino acid sequence with at least 95%, 98%, 99%, or 100% identity to SEQ ID NO:4; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:3 and second amino acid sequence with 100% identity to SEQ ID NO:4, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:3 and a second amino acid sequence with 100% identity to SEQ ID NO:4. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, 10, 11, 12, 13, 14, or 15), or all, of determinative parameter numbers 3, 4, 5, 13, 16, 17, 19, 29, 30, 31, 32, 33, 34, 36, and/or 37 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (including 1, 2, 3, 4, 5, 6, 7, 8, 9, 10, 11, 12, 13, 14, or 15), or all, of determinative parameter numbers 3, 4, 5, 13, 16, 17, 19, 29, 30, 31, 32, 33, 34, 36, and/or 37 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein such methods include: providing a sample of a test recombinant antibody preparation having a first amino acid sequence with at least 95%, 98%, 99%, or 100% identity to SEQ ID NO:5 and a second amino acid sequence with at least 95%, 98%, 99%, or 100% identity to SEQ ID NO:6; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:5 and second amino acid sequence with 100% identity to SEQ ID NO:6, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:5 and a second amino acid sequence with 100% identity to SEQ ID NO:6. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, or 10), or all, of determinative parameter numbers 13, 8, 19, 12, 14, 15, 29, 30, 31, and/or 34 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (1, 2, 3, 4, 5, 6, 7, 8, 9, or 10), or all, of determinative parameter numbers 13, 8, 19, 12, 14, 15, 29, 30, 31, and/or 34 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein the methods include: providing a sample of a test recombinant antibody preparation having a first amino acid sequence with at least 95%, 98%, 99%, or 100% identity to SEQ ID NO:7 and a second amino acid sequence with at least 95%, 98%, 99%, or 100% identity to SEQ ID NO:8; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:7 and second amino acid sequence with 100% identity to SEQ ID NO:8, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:7 and a second amino acid sequence with 100% identity to SEQ ID NO:8. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, 10, 11, 12, 13, 14, 15, 16, 17, 18, 19), or all, of determinative parameter numbers 4, 6, 25, 26, 27, 28, 13, 8, 19, 11, 12, 14, 15, 18, 29, 30, 31, 36, and/or 37 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (1, 2, 3, 4, 5, 6, 7, 8, 9, 10, 11, 12, 13, 14, 15, 16, 17, 18, 19), or all, of determinative parameter numbers 4, 6, 25, 26, 27, 28, 13, 8, 19, 11, 12, 14, 15, 18, 29, 30, 31, 36, and/or 37 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein the methods include: providing a sample of a test recombinant antibody preparation having a first amino acid sequence with at least 95%, 98%, 99%, or 100% identity to SEQ ID NO:9 and a second amino acid sequence with at least 95%, 98%, 99%, or 100% identity to SEQ ID NO:10; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:9 and second amino acid sequence with 100% identity to SEQ ID NO:10, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:9 and a second amino acid sequence with 100% identity to SEQ ID NO:10. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, 10, 11, 12, 13, 14, 15, 16, 17, 18, 19, 20, 21, or 22), or all, of determinative parameter numbers 5, 25, 26, 27, 28, 13, 16, 17, 19, 14, 10, 15, 18, 29, 30, 31, 32, 34, 36, 37, 39, and/or 40 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (including 1, 2, 3, 4, 5, 6, 7, 8, 9, 10, 11, 12, 13, 14, 15, 16, 17, 18, 19, 20, 21, or 22), or all, of determinative parameter numbers 5, 25, 26, 27, 28, 13, 16, 17, 19, 14, 10, 15, 18, 29, 30, 31, 32, 34, 36, 37, 39, and/or 40 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein the methods include: providing a sample of a test recombinant antibody preparation having a first amino acid sequence with at least 95%, 98%, 99%, or 100% identity to SEQ ID NO:11 and a second amino acid sequence with at least 95%, 98%, 99%, or 100% identity to SEQ ID NO:12; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:11 and second amino acid sequence with 100% identity to SEQ ID NO:12, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:11 and a second amino acid sequence with 100% identity to SEQ ID NO:12. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, 10, 11, 12, 13, 14, or 15), or all, of determinative parameter numbers 4, 6, 13, 16, 17, 19, 11, 20, 21 or 22, 29, 30, 31, 32, 34, and/or 38 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (including 1, 2, 3, 4, 5, 6, 7, 8, 9, 10, 11, 12, 13, 14, or 15), or all, of determinative parameter numbers 4, 6, 13, 16, 17, 19, 11, 20, 21 or 22, 29, 30, 31, 32, 34, and/or 38 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein the methods include: providing a sample of a test recombinant antibody preparation having a first amino acid sequence with at least 95%, 98%, 99%, or 100% identity to SEQ ID NO:13 and a second amino acid sequence with at least 95%, 98%, 99%, or 100% identity to SEQ ID NO:14; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:13 and second amino acid sequence with 100% identity to SEQ ID NO:14, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:13 and a second amino acid sequence with 100% identity to SEQ ID NO:14. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, 10, 11, 12, 13, 14, or 15), or all, of determinative parameter numbers 3, 4, 13, 16, 17, 9, 19, 8, 10, 20, 29, 30, 31, 32, and/or 35 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, the at least one input value comprises one or more, at least one (including 1, 2, 3, 4, 5, 6, 7, 8, 9, 10, 11, 12, 13, 14, or 15), or all, of determinative parameter numbers 3, 4, 13, 16, 17, 9, 19, 8, 10, 20, 29, 30, 31, 32, and/or 35 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In some aspects, the disclosure provides methods of manufacturing a pharmaceutical product comprising a recombinant antibody, wherein the methods include: providing a sample of a test recombinant antibody preparation having a first amino acid sequence with at least 95%, 98%, 99%, or 100% identity to SEQ ID NO:15 and a second amino acid sequence with at least 95%, 98%, 99%, or 100% identity to SEQ ID NO:16; acquiring an input value for each of a plurality of parameters in the test recombinant antibody preparation, wherein one or more of the plurality are determinative parameters; acquiring a plurality of assessments made by comparing the input value with a plurality of target values for a target protein having a first amino acid sequence with 100% identity to SEQ ID NO:15 and second amino acid sequence with 100% identity to SEQ ID NO:16, wherein the target protein is approved under a biologics license application (BLA) or a supplemental BLA; and processing the test recombinant antibody preparation into a pharmaceutical product comprising a recombinant antibody if the input values for at least one of the one or more determinative parameters are indistinguishable from the target values for said one or more determinative parameters for the target protein, wherein the recombinant antibody preparation is not approved under a BLA or supplemental BLA. In some instances, the test recombinant antibody preparation comprises a first amino acid sequence with 100% identity to SEQ ID NO:15 and a second amino acid sequence with 100% identity to SEQ ID NO:16. In some instances, the acquiring step comprises acquiring an input value for a plurality of determinative entries and the formulating step comprises formulating the test recombinant antibody preparation into a pharmaceutical product comprising a recombinant antibody preparation if the input values for the plurality of determinative parameters are indistinguishable from the target values for said plurality of determinative parameters for the target protein. In some instances, the determinative parameter(s) comprise one or more, at least one (including 1, 2, 3, 4, 5, 6, 7, 8, 9, 10, 11, or 12), or all, of determinative parameter numbers 3, 26, 13, 8, 11, 9, 10, 29, 30, 31, 35, and/or 33 shown in Table 2. In some instances, the recombinant antibody preparation is approved under Section 351(k) of the Public Health Service (PHS) Act. In some instances, the test recombinant antibody preparation is drug substance. In some instances, the test recombinant antibody preparation is drug product. In some instances, at least one input value is directly obtained. In some instances, at least one input value comprises one or more, at least one (including 1, 2, 3, 4, 5, 6, 7, 8, 9, 10, 11, or 12), or all, of determinative parameter numbers 3, 26, 13, 8, 11, 9, 10, 29, 30, 31, 35, and/or 33 shown in Table 2. In some instances, the at least one input value is directly obtained using a method provided in TABLE 3. In some instances, processing the test recombinant antibody preparation into a pharmaceutical product comprising a recombinant antibody preparation comprises combining the test antibody preparation with an excipient or buffer. In some instances, processing the test recombinant antibody preparation into a pharmaceutical product comprising a recombinant antibody preparation comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location. In some instances, the step of providing a sample of a test recombinant antibody preparation comprises expressing the test recombinant antibody preparation.

In one embodiment, in any of the aspects described herein, at least one input value is acquired by performing an analytical analysis on said test biologic sample. In one embodiment, the method is implemented on a computer.

### DEFINITIONS

As used herein, the terms biologics, biotherapeutics, and biologic products are used interchangeably to refer to peptide and protein products. For example, biologics herein include naturally derived or recombinant products expressed in cells, such as, e.g., proteins, glycoproteins, fusion proteins, growth factors, vaccines, blood factors, thrombolytic agents, hormones, interferons, interleukin based products, monospecific (e.g., monoclonal) antibodies, therapeutic enzymes. Some biologics are approved under a biologics license application (BLA), under section 351(a) of the Public Health Service (PHS) Act, whereas biosimilar and interchangeable biologics referencing a BLA as a reference product are licensed under section 351(k) of the PHS Act. Section 351 of the Public Health Service (PHS) Act is codified as 42 U.S.C. 262. Other biologics may be approved under section 505(b)(1) of the Federal Food and Cosmetic Act, or as abbreviated applications under sections 505(b)(2) and 505(j) of the Hatch Waxman Act, wherein section 505 is codified 21 U.S.C. 355.

A used herein, approval refers to the procedure by which a regulatory entity, e.g., the FDA or EMEA, approves a candidate for therapeutic or diagnostic use in humans or animals. As used herein, a primary approval process is an approval process which does not refer to a previously approved protein, e.g., it does not require that the protein being approved have structural or functional similarity to a previously approved protein, e.g., a previously approved protein having the same primary amino acid sequence or a primary amino acid sequence that differs by no more than 1, 2, 3, 4, 5, or 10 residues or that has 98% or more sequence identity. In embodiments the primary approval process is one in which the applicant does not rely, for approval, on data, e.g., clinical data, from a previously approved product. Exemplary primary approval processes include, in the U.S, a Biologics License Application (BLA), or supplemental Biologics License Application (sBLA), a new drug application (NDA) under 505(b)(1) of the Federal Food and Cosmetic Act, and in Europe an approval in accordance with the provisions of Article 8(3) of the European Directive 2001/83/EC, or an analogous proceeding in other countries or jurisdictions.

As used herein, a secondary approval process is an approval process which refers to clinical data for a previously approved product. In embodiments the secondary approval requires that the product being approved have structural or functional similarity to a previously approved product, e.g., a previously approved protein having the same primary amino acid sequence or a primary amino acid sequence that differs by no more than 1, 2, 3, 4, 5, or 10 residues or that has at least 98%, 99% or more (100%) sequence identity. In embodiments the secondary approval process is one in which the applicant relies, for approval, on clinical data from a previously approved product. Exemplary secondary approval processes include, in the U.S, an approval under 351(k) of the Public Health Service Act or under section 505(j) or 505(b)(2) of the Hatch Waxman Act and in Europe, an application in accordance with the provisions of Article 10, e.g., Article 10(4), of the European Directive 2001/83/EC, or an analogous proceeding in other countries or jurisdictions.

As used herein, a glycoprotein refers to amino acid sequences that include one or more oligosaccharide chains (e.g., glycans) covalently attached thereto. Exemplary amino acid sequences include peptides, polypeptides and proteins. Exemplary glycoproteins include glycosylated antibodies and antibody-like molecules (e.g., Fc fusion proteins). Exemplary antibodies include monoclonal antibodies and/or fragments thereof, polyclonal antibodies and/or fragments thereof, and Fc domain containing fusion proteins (e.g., fusion proteins containing the Fc region of IgG1, or a glycosylated portion thereof). A glycoprotein preparation is a composition or mixture that includes at least one glycoprotein.

In some embodiments, a glycoprotein preparation (e.g., such as a glycoprotein drug substance or a precursor thereof) can be a sample from a proposed or test batch of glycoprotein drug substance or drug product. As used herein, a batch of a glycoprotein preparation refers to a single production run of the glycoprotein. Evaluation of different batches thus means evaluation of different production runs or batches. As used herein sample(s) refer to separately procured samples. For example, evaluation of separate samples could mean evaluation of different commercially available containers or vials of the same batch or from different batches.

As used herein, target biologic, e.g., target protein, refers to a commercially available, or approved, biologic which defines or provides the basis against which a test biologic is measured or evaluated. In embodiments a target biologic is commercially available for therapeutic use in humans or animals. In embodiments the target biologic was approved for use in humans or animals by a primary approval process. In embodiments the target biologic is a reference listed drug for a secondary approval process. Examples of proteins that are target proteins in the United States include those in Table 1A and Table 1B herein. An exemplary target protein is an antibody, e.g., a CDR-grafted, humanized or human antibody. Other target proteins include glycoproteins, cytokines, hematopoietic proteins, soluble receptor fragments, and growth factors.

As used herein, a non-test biologic, e.g., a non-test protein, is a biologic other than the test biologic. In embodiments a non-test protein is a member of a class of proteins that includes the test protein. For example, the test protein and the non-test protein are both antibodies. In embodiments both the test protein and the non-test protein are members of the same class of antibodies e.g., both are IgG or both are IgM antibodies. In embodiments both are Fc-containing proteins, e.g., Fc fusion proteins. In embodiments both the test protein and the non-test protein are CDR-grafted antibodies, humanized antibodies, or human antibodies. In embodiments the non-test protein is an approved protein, e.g., a protein approved by a primary approval process. In embodiments the non-test protein is an approved antibody, e.g., an antibody approved by a primary approval process. As used herein, a plurality of non-test proteins includes X non-test proteins, wherein X is, equal to, at least, or more than, 2, 3, 4, 5, 10, or 15. In a plurality of non-test proteins, one, more than one, e.g., 2, 3, 4, 5, or 6, or all of the non-test proteins are: members of a class of proteins that includes the test protein; antibodies; antibodies of the same class, e.g., IgG or IgM antibodies; CDR-grafted antibodies; humanized antibodies; human antibodies; Fc-containing proteins, e.g., Fc fusion proteins; approved proteins, e.g., proteins, e.g., antibodies, approved by a primary approval process.

As used herein, evaluating, e.g., in the evaluation/evaluating, identifying, and/or producing methods disclosed herein means reviewing, considering, determining, assessing, measuring, and/or detecting the presence, absence, level, and/or ratio of one or more parameters in a test and/or target biologic to provide information pertaining to the one or more parameters. In some instances, evaluating a glycoprotein preparation includes detecting the presence, absence, level or ratio of one or more (e.g., two or more when working with ratios) disclosed in Table 1 using methods disclosed in Table 3.

As used herein, acquire or acquiring refers to obtaining possession of a physical entity, or a value, e.g., a numerical value, by “directly acquiring” or “indirectly acquiring” the physical entity or value. “Directly acquiring” means performing a process (e.g., performing an assay or test on a sample or “analyzing a sample” as that term is defined herein) to obtain the physical entity or value. “Indirectly acquiring” refers to receiving the physical entity or value from another party or source (e.g., a third party laboratory that directly acquired the physical entity or value). Directly acquiring a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, analyzing a sample includes performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Analyzing a sample can include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, a parameter associated with a test biologic, e.g., protein, e.g., an antibody, refers to a characteristic associated with the test biologic (e.g., a characteristic associated with a moiety of a test biologic). In embodiments the moiety is part of the test biologic, e.g., connected with the rest of the test biologic by a covalent bond, and the parameter is referred to herein as an intrinsic parameter. Intrinsic parameters include the presence, absence, level, ratio (with another entity), or distribution of a physical moiety, e.g., a moiety arising from or associated with a post-translational event. Exemplary parameters of this type include the presence, absence, level, ratio (with another entity), or distribution of a glycan or glycoform described herein. In embodiments the moiety is not part of the test biologic but is present in the sample with the test biologic and the parameter is referred to herein as a sample, or extrinsic, parameter. Exemplary parameters of this type include the presence, absence, level, ratio (with another entity), or distribution of impurities, e.g., whole cell proteins, residue from purification processes, viral components, and enclosure components. The presence, absence, level, ratio (with another entity), distribution of misfolded or denatured product is a sample or extrinsic parameter.

As used herein, a determinative parameter is a parameter that defines a target biologic and can distinguish a test biologic from a plurality of non-test biologics, e.g., relative to a target biologic, and support a determination of sameness or identity of the test biologic with a target biologic (see section entitled “Determinative and Non-determinative test protein parameters”).

As used herein, a signature comprises a plurality of determinative test biologic parameters (or the input values therefor). In an embodiment the signature includes X determinative test biologic parameters, wherein X is, equal to, at least, or greater than, 2, 3, 4, 5, 6, 7, 8, 9, 10, 15, 20, 25, 30, 50, 75, 100 or more.

As used herein, an input value is a value associated with a parameter of a test biologic. The value can be qualitative, e.g., present, absent, intermediate, or the value can be qualitative, e.g., it can be a numerical value such as a single number, or a range, for a parameter.

### BRIEF DESCRIPTION OF THE FIGURES

FIG. 1 shows an amino acid sequence of a heavy chain of a recombinant antibody (SEQ ID NO:1).

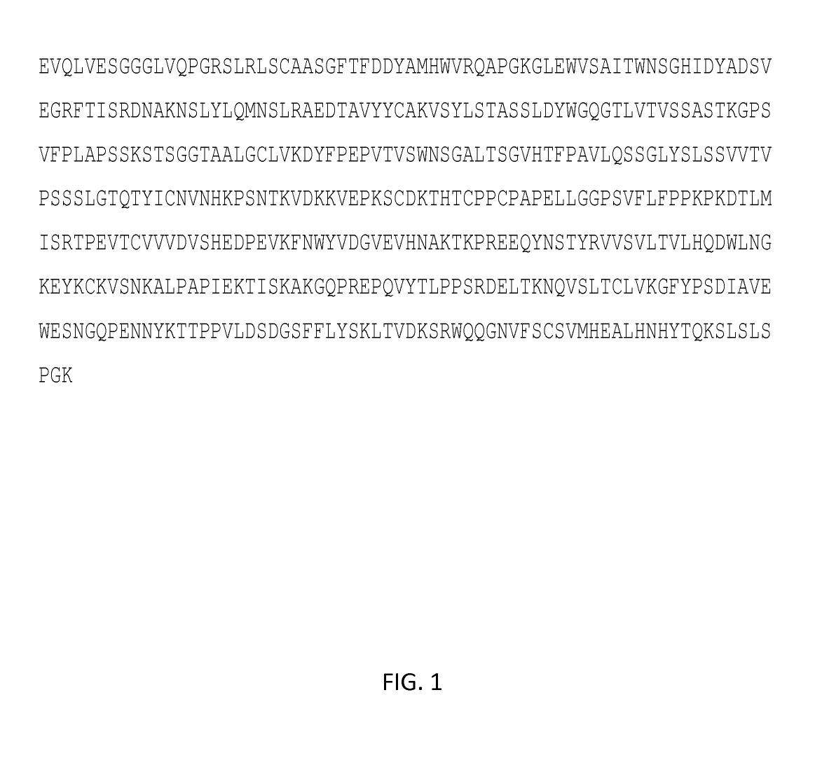

FIG. 2 shows an amino acid sequence of a light chain of a recombinant antibody (SEQ ID NO:2).

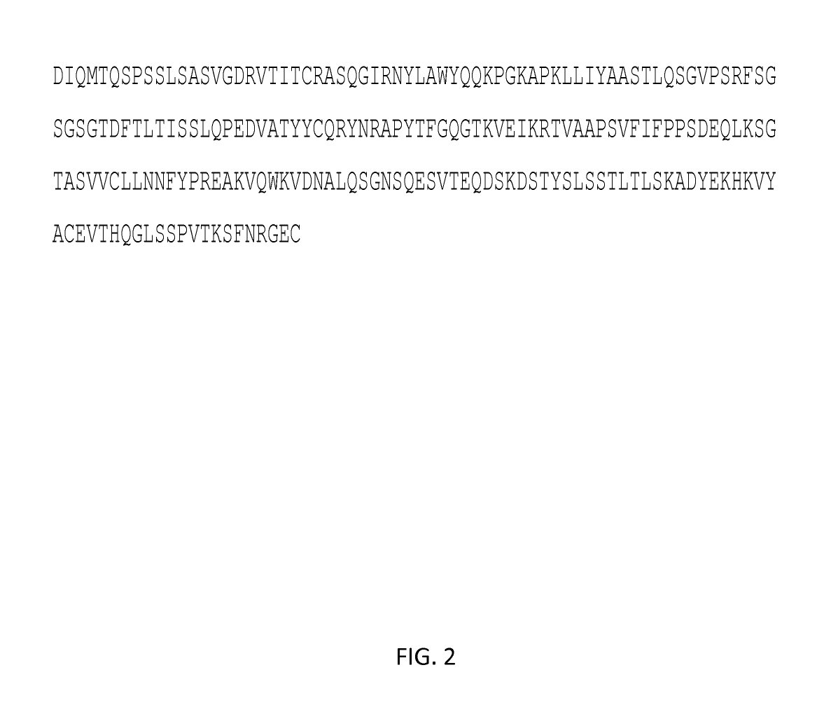

FIG. 3 shows an amino acid sequence of a heavy chain of a recombinant antibody (SEQ ID NO: 3).

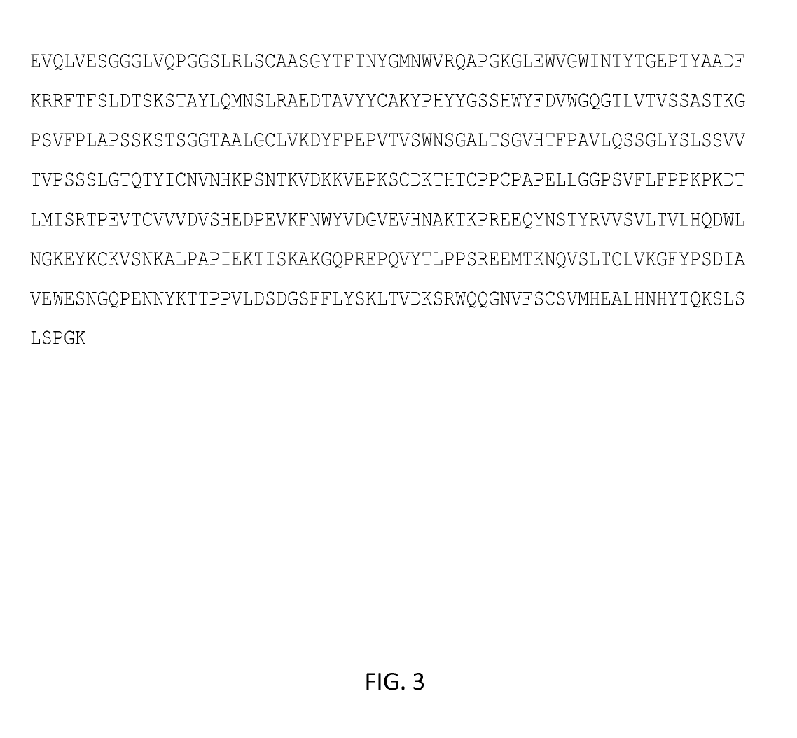

FIG. 4 shows an amino acid sequence of a light chain of a recombinant antibody (SEQ ID NO:4).

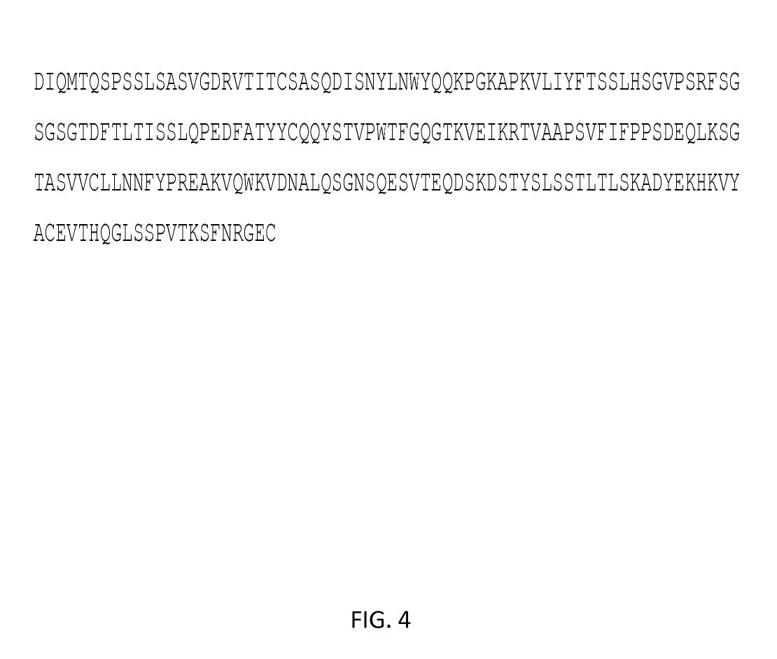

FIG. 5 shows an amino acid sequence of a heavy chain of a recombinant antibody (SEQ ID NO:5).

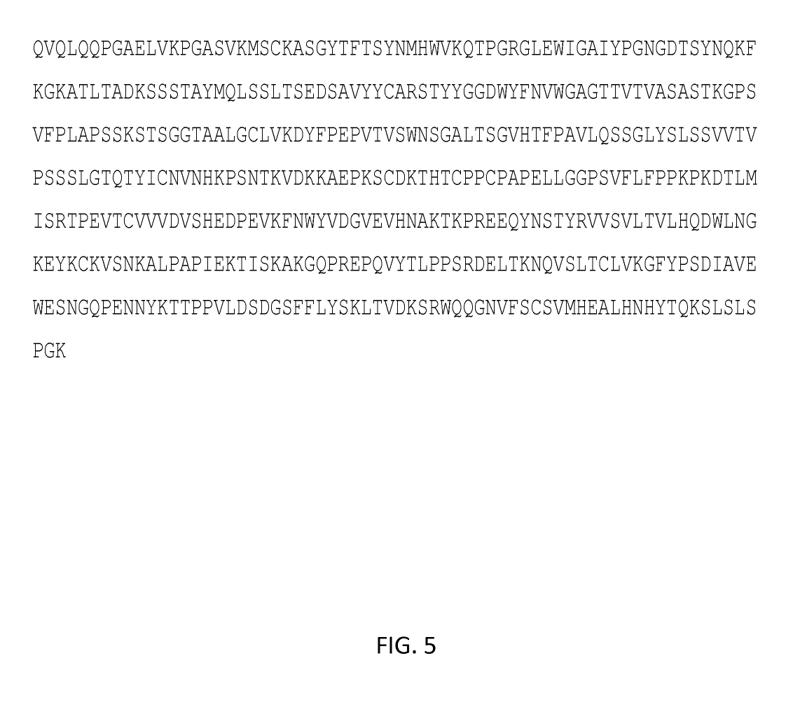

FIG. 6 shows an amino acid sequence of a light chain of a recombinant antibody (SEQ ID NO:6).

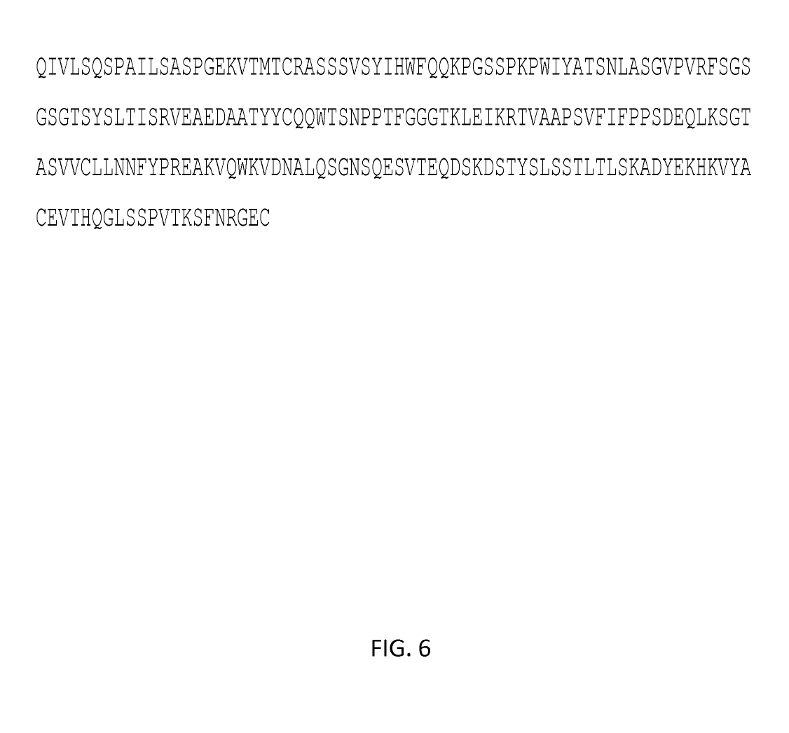

FIG. 7 shows an amino acid sequence of a heavy chain of a recombinant antibody (SEQ ID NO:7).

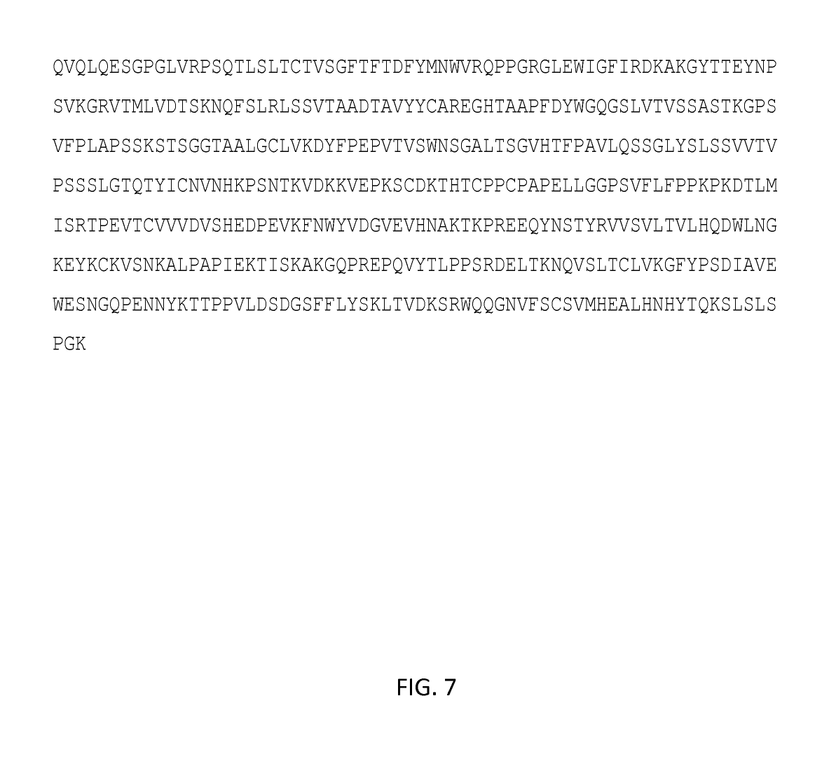

FIG. 8 shows an amino acid sequence of a light chain of a recombinant antibody (SEQ ID NO:8).

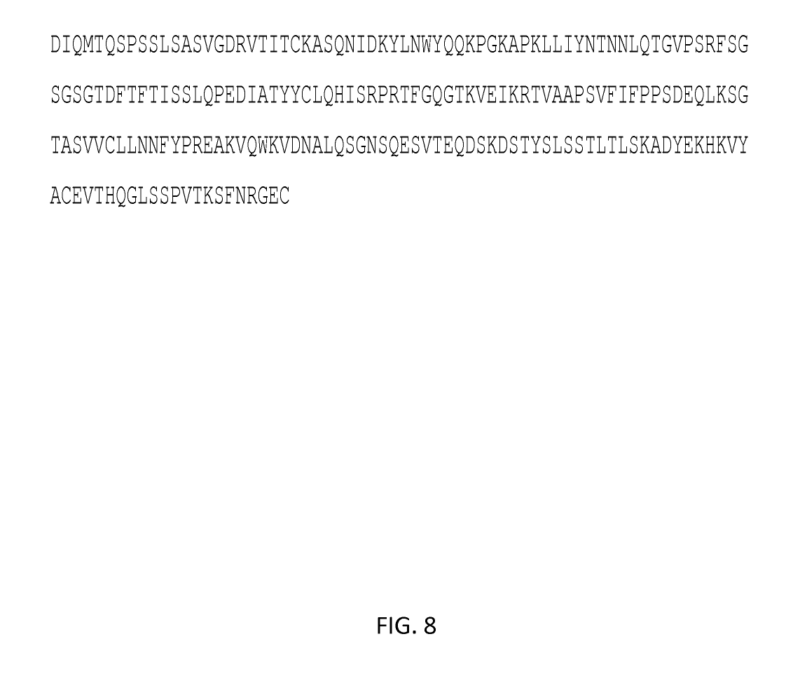

FIG. 9 shows an amino acid sequence of a heavy chain of a recombinant antibody (SEQ ID NO:9).

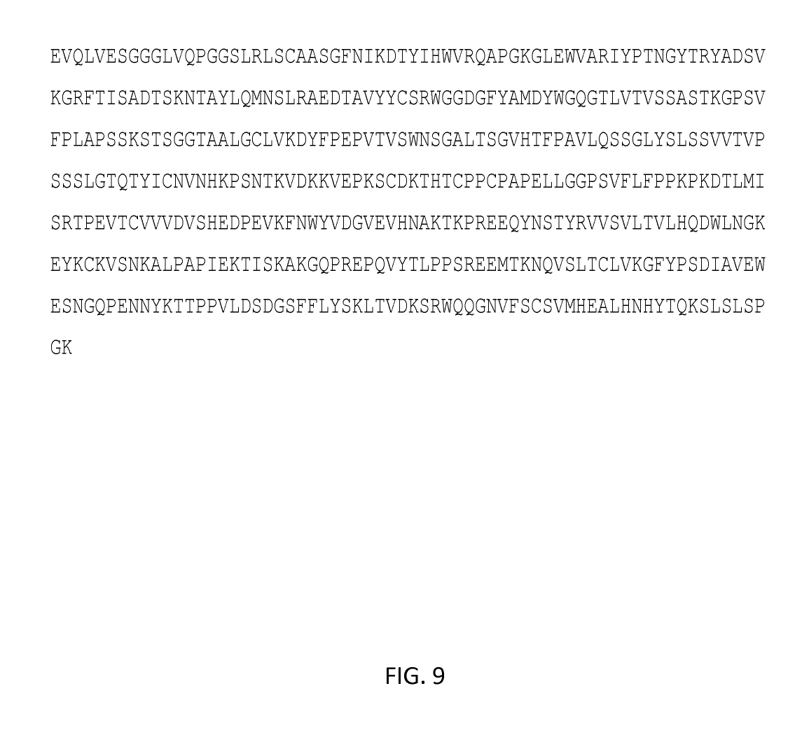

FIG. 10 shows an amino acid sequence of a light chain of a recombinant antibody (SEQ ID NO:10).

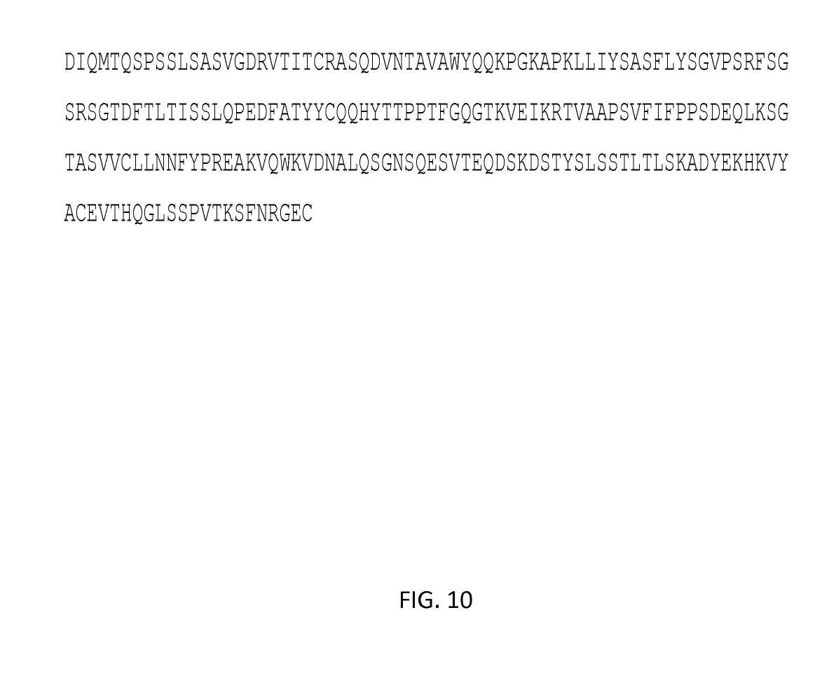

FIG. 11 shows an amino acid sequence of a heavy chain of a recombinant antibody (SEQ ID NO:11).

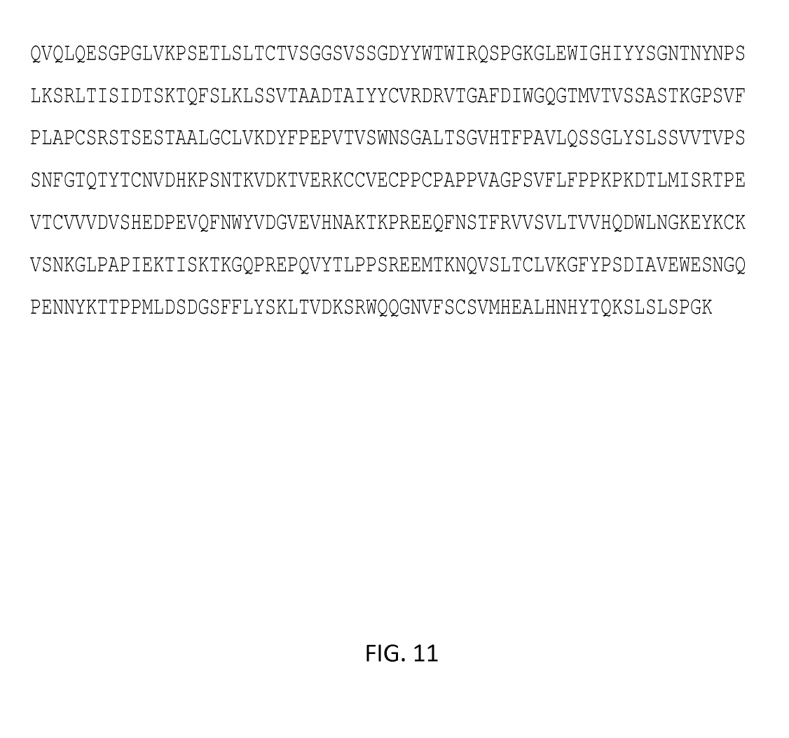

FIG. 12 shows an amino acid sequence of a light chain of a recombinant antibody (SEQ ID NO:12).

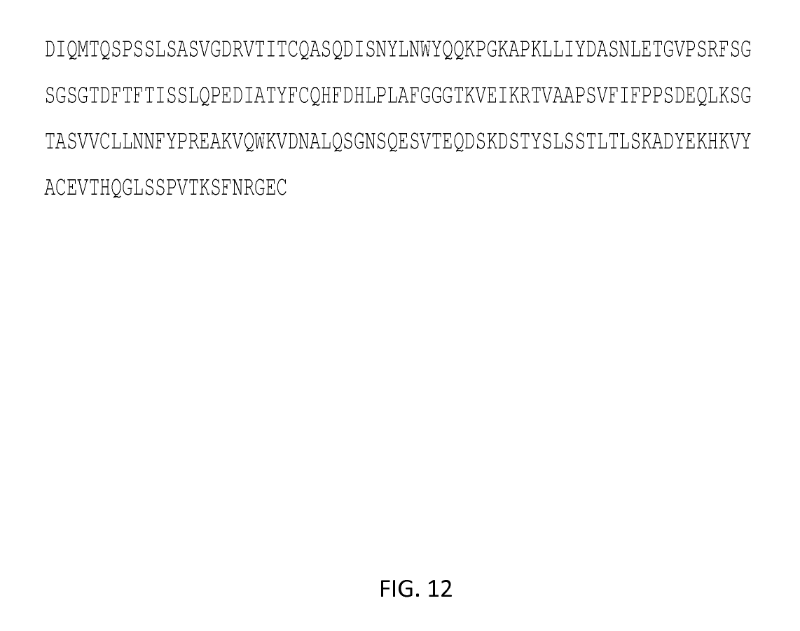

FIG. 13 shows an amino acid sequence of a heavy chain of a recombinant antibody (SEQ ID NO:13).

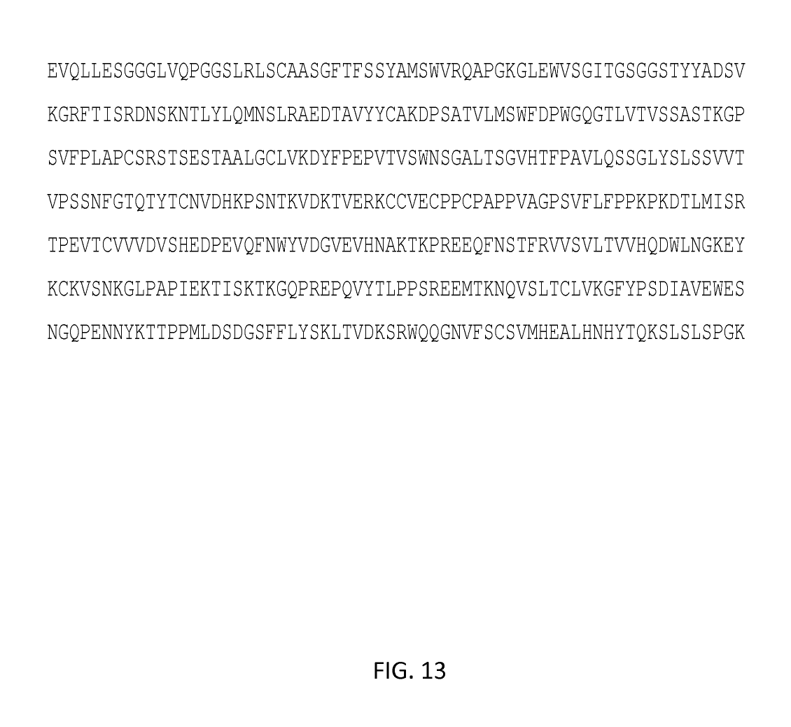

FIG. 14 shows an amino acid sequence of a light chain of a recombinant antibody (SEQ ID NO:14).

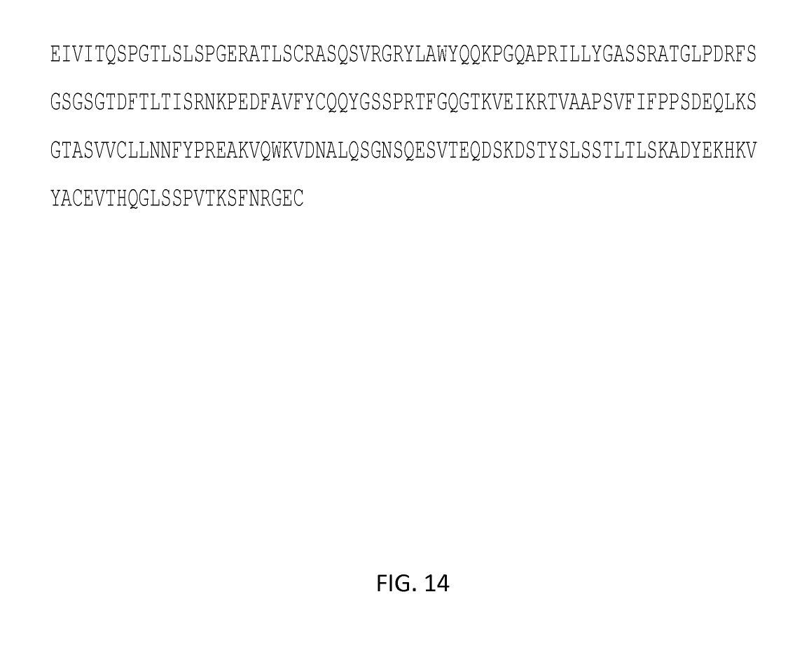

FIG. 15 shows an amino acid sequence of a heavy chain of a recombinant antibody.

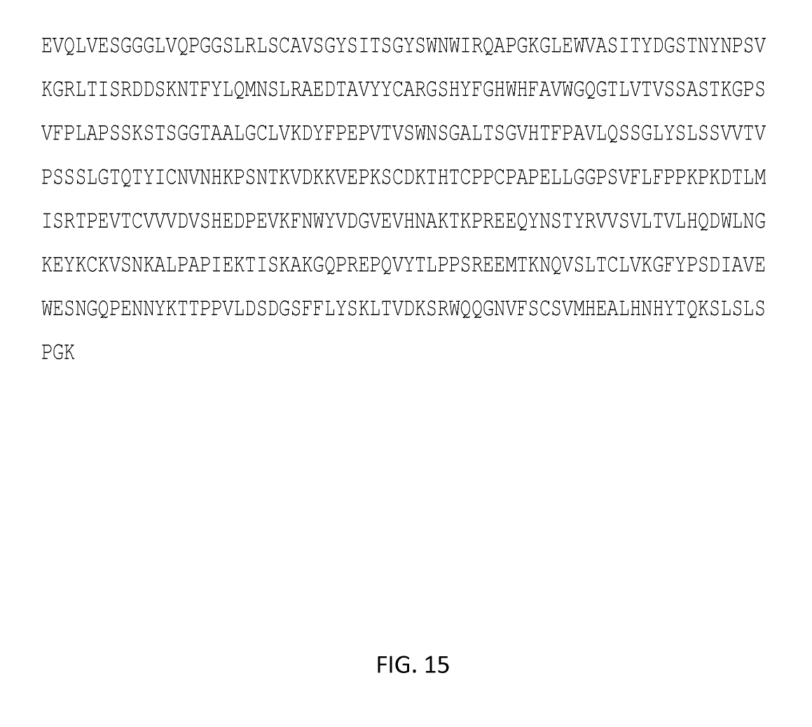

FIG. 16 shows an amino acid sequence of a light chain of a recombinant antibody.

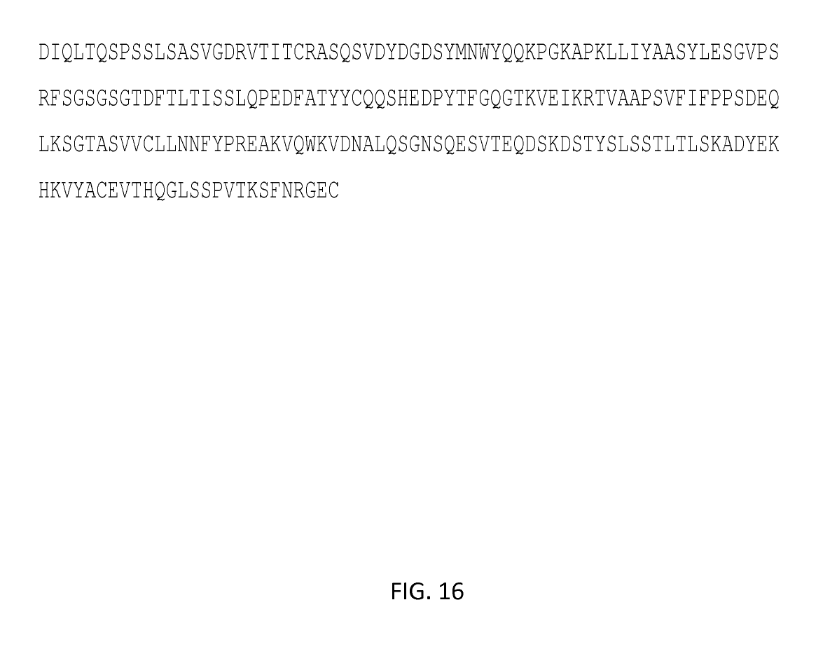

### DETAILED DESCRIPTION

Relevant literature suggests that information necessary to make and test true generic biologics, including, for example, biosimilars and interchangeables, is unavailable (see, e.g., Nowicki, “Basic Facts about Biosimilars,” Kidney Blood Press. Res., 30:267-272 (2007); Hincal “An Introduction To Safety Issues In Biosimilars/Follow-On Biopharmaceuticals”, J. Med. CBR Def., 7:1-18, (2009); Roger, “Biosimilars: current status and future directions,” Expert Opin. Biol. Ther., 10(7):1011-1018 (2010)). One exemplary report states that “[t]he size and complexity of . . . therapeutic proteins make the production of an exact replica almost impossible; therefore, there are no true generic forms of these proteins . . . [v]erification of the similarity of biosimilars to innovator medicines remains a key challenge” (Hincal, supra). Accordingly, the science and technology for establishing biosimilarity is fundamentally different from the science and technology required for developing novel biological products.

### Test Proteins and Target Proteins

Methods described herein can be used to make and/or evaluate a test biologic preparation, e.g., a test protein preparation.

A test biologic refers to the biologic, e.g., protein, being evaluated for similarity to a target biologic, e.g., a target protein. The test biologic may or may not be commercially available. In embodiments a test biologic is not commercially available for therapeutic use in humans or animals. In an embodiment the test biologic has not been approved for therapeutic or diagnostic use in humans or animals. In an embodiment the test biologic has been approved, e.g., under a secondary approval process, for therapeutic or diagnostic use in humans or animals. In embodiments, a test biologic has the same primary amino acid sequence as a target protein or will differ by no more than 1, 2, 3, 4, 5, 10, 15, 20, 25, 30 residues or has at least 90, 95, 98, 99% or is identical to a target biologic sequence.

The terms the same primary amino acid sequence, a primary amino acid sequence that differs by no more than 1, 2, 3, 4, 5, 10, 15, 20, 25, or 30 residues, sequences that have at least 98% or more sequence identity, or similar terms, relate to the level of identity between the primary amino acid sequence, e.g., of first protein, e.g., a test protein, and the primary amino acid sequence, e.g., of second protein, e.g., a target protein. In some embodiments a product will include amino acid variants, e.g., species that differ at terminal residues, e.g., at one or two terminal residues. In embodiments of such cases, the sequence identity compared is the identity between the primary amino acid sequence of the most abundant active species in each of the products being compared. In some embodiments sequence identity refers to the amino acid sequence encoded by a nucleic acid that can be used to make the product.

### Antibodies

In some embodiments, biologics include glycoproteins, e.g., such as antibodies, e.g., monospecific antibodies, e.g., monoclonal antibodies. The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized are preferred

As used herein, an “immunoglobulin variable domain sequence” refers to an amino acid sequence which can form the structure of an immunoglobulin variable domain such that one or more CDR regions are positioned in a conformation suitable for an antigen binding site.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883.

The antibody can be, e.g., a CDR-grafted antibody, a humanized antibody or a human antibody. A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

The methods described herein include, inter alia, processing a test protein preparation, e.g., an antibody test protein preparation, if the input value or input values of test protein meet a predetermined threshold for sameness with a predefined plurality of target values for said determinative test protein parameters for a target protein, e.g., the input values for determinative test protein parameters are indistinguishable from target protein values for the determinative test protein parameters.

The target proteins are commercially available, or approved, proteins which define or provide the basis against which a test protein is measured or evaluated. Examples of target antibodies that have obtained regulatory approval, e.g., under a BLA, a supplemental BLA, or equivalent thereof, include the following:

TABLE 1A

| | Brand | Approval | | | Approved |
|---|---|---|---|---|---|
| Antibody | name | date | Type | Target | Indication |
| Muromonab- | Orthoclone | 1986 | murine | T cell CD3 | Transplant rejection |
| CD3 | OKT3 | | | Receptor | |
| Abciximab | ReoPro | 1994 | chimeric | inhibition of | Cardiovascular |
| | | | | glycoprotein | disease |
| | | | | IIb/IIIa | |
| Daclizumab | Zenapax | 1997 | humanized | IL-2Rα receptor | Transplant rejection |
| | | | | (CD25) | |
| Rituximab | Rituxan, | 1997 | chimeric | CD20 | Non-Hodgkin |
| | Mabthera | | | | lymphoma |
| Basiliximab | Simulect | 1998 | chimeric | IL-2Rα receptor | Transplant rejection |
| | | | | (CD25) | |
| Infliximab | Remicade | 1998 | chimeric | inhibition of TNF-α | Several autoimmune |
| | | | | signaling | disorders |
| Palivizumab | Synagis | 1998 | humanized | an epitope of the | Respiratory Syncytial |
| | | | | RSV F protein | Virus |
| Trastuzumab | Herceptin | 1998 | humanized | ErbB2 | Breast cancer |
| Gemtuzumab | Mylotarg | 2000 | humanized | CD33 | Acute myelogenous |
| | | | | | leukemia (with |
| | | | | | calicheamicin) |
| Alemtuzumab | Campath | 2001 | humanized | CD52 | Chronic lymphocytic |
| | | | | | leukemia |
| Efalizumab | Raptiva | 2002 | humanized | CD11a | Psoriasis |
| Adalimumab | Humira | 2002 | human | inhibition of TNF-α | Several auto-immune |
| | | | | signaling | disorders |
| Ibritumomab | Zevalin | 2002 | murine | CD20 | Non-Hodgkin |
| tiuxetan | | | | | lymphoma (with |
| | | | | | yttrium-90 or indium- |
| | | | | | 111) |
| Tositumomab | Bexxar | 2003 | murine | CD20 | Non-Hodgkin |
| | | | | | lymphoma |
| Cetuximab | Erbitux | 2004 | chimeric | epidermal growth | Colorectal cancer, |
| | | | | factor receptor | Head and neck cancer |
| Bevacizumab | Avastin | 2004 | humanized | Vascular | Colorectal cancer, |
| | | | | endothelial growth | Age related macular |
| | | | | factor (VEGF) | degeneration (off- |
| | | | | | label) |
| Omalizumab | Xolair | 2004 | humanized | immunoglobulin E | mainly allergy- |
| | | | | (IgE) | related asthma |
| Natalizumab | Tysabri | 2006 | humanized | alpha-4 (α4) | Multiple sclerosis |
| | | | | integrin, | and Crohn's disease |
| Ranibizumab | Lucentis | 2006 | humanized | Vascular | Macular degeneration |
| | | | | endothelial growth | |
| | | | | factor A (VEGF-A) | |
| Panitumumab | Vectibix | 2006 | human | epidermal growth | Colorectal cancer |
| | | | | factor receptor | |
| Eculizumab | Soliris | 2007 | humanized | Complement | Paroxysmal nocturnal |
| | | | | system protein C5 | hemoglobinuria |
| Certolizumab | Cimzia | 2008 | humanized | inhibition of TNF-α | Crohn's disease |
| pegol^{[19]} | | | | signaling | |
| Canakinumab | Ilaris | 2009 | Human | IL-1β | Cryopyrin-associated |
| | | | | | periodic syndromes |
| | | | | | (CAPS) |
| Ofatumumab | Arzerra | 2009 | Human | CD20 | Chronic lymphocytic |
| | | | | | leukemia |
| Golimumab | Simponi | 2009 | Human | TNF-alpha | Rheumatoid arthritis, |
| | | | | inihibitor | Psoriatic arthritis, and |
| | | | | | Ankylosing |
| | | | | | spondylitis |
| Denosumab | Prolia, | 2010 | Human | RANK Ligand | Postmenopausal |
| | Xgeva | | | inhibitor | osteoporosis, aolid |
| | | | | | tumor's bony |
| | | | | | metasteses |
| Tocilizumab | Actemra and | 2010 | Humanised | Anti-IL-6R | Rheumatoid arthritis |
| (or Atlizumab) | RoActemra | | | | |
| Belimumab | Benlysta | 2011 | human | inihibition of B- | Systemic lupus |
| | | | | cell activating | erythematosus |
| | | | | factor | |
| Brentuximab | Adcetris | 2011 | Chimeric | CD30 | Anaplastic large cell |
| vedotin | | | | | lymphoma (ALCL) |
| | | | | | and Hodgkin |
| | | | | | lymphoma |
| Ipilimumab | Yervoy | 2011 | Human | blocks CTLA-4 | Melanoma |
| (MDX-101) | | | | | |

Other products that can be target proteins in the methods described herein include those in Table 1B:

TABLE 1B

| Protein Product | Brand name of Reference Drug |
|---|---|
| interferon gamma-1b | Actimmune ® |
| +alteplase; tissue plasminogen activator | Activasec ®)/Cathflo ® |
| Recombinant antihemophilic factor | Advate |
| human albumin | Albutein ® |
| laronidase | Aldurazyme ® |
| interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein | Amevive ® |
| LFA3-Ig | |
| bivalirudin | Angiomax ® |
| darbepoetin alfa | Aranesp ™ |
| interferon beta-1a; recombinant | Avonex ® |
| coagulation factor IX | BeneFix ™ |
| Interferon beta- lb | Betaseron ® |
| antihemophilic factor | Bioclate ™ |
| human growth hormone | BioTropin ™ |
| botulinum toxin type A | Botox ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune Fab, ovine | DigiFab ™ |
| rasburicase | Elitek ® |
| etanercept | Enbrel ® |
| epoietin alfa | Epogen ® |
| algasidase beta | Fabrazyme ® |
| urofollitropin | Fertinex ™ |
| follitropin beta | Follistim ™ |
| teriparatide | Forteo ® |
| human somatropin | GenoTropin ® |
| glucagon | GlucaGen ® |
| follitropin alfa | Gonal-F ® |
| antihemophilic factor | Helixate ® |
| Antihemophilic Factor; Factor XIII | Hemofil ® |
| insulin | Humalog ® |
| antihemophilic factor/von Willebrand factor | Humate-P ® |
| complex-human | |
| somatotropin | Humatrope ® |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| palifermin | Kepivance |
| anakinra | Kineret ™ |
| antihemophilic factor | Kogenate ® FS |
| insulin glargine | Lantus ® |
| granulocyte macrophage colony-stimulating | Leukine ®/Leukine ® Liquid |
| factor | |
| lutropin alfa, for injection | Luveris |
| OspA lipoprotein | LYMErix ™ |
| galsulfase | Naglazyme ™ |
| nesiritide | Natrecor ® |
| pegfilgrastim | Neulasta ™ |
| oprelvekin | Neumega ® |
| filgrastim | Neupogen ® |
| fanolesomab | NeutroSpec ™(formerly LeuTech ®) |
| somatropin [rDNA] | Norditropin ®/Norditropin Nordiflex ® |
| insulin; zinc suspension; | Novolin L ® |
| insulin; isophane suspension | Novolin N ® |
| insulin, regular; | Novolin R ® |
| insulin | Novolin ® |
| coagulation factor VIIa | NovoSeven ® |
| somatropin | Nutropin ® |
| immunoglobulin intravenous | Octagam ® |
| PEG-L-asparaginase | Oncaspar ® |
| abatacept, fully human soluable fusion protein | Orencia ™ |
| human chorionic gonadotropin | Ovidrel ® |
| peginterferon alfa-2a | Pegasys ® |
| pegylated version of interferon alfa-2b | PEG-Intron ™ |
| Abarelix (injectable suspension); gonadotropin- | Plenaxis ™ |
| releasing hormone antagonist | |
| epoietin alfa | Procrit ® |
| aldesleukin | Proleukin, IL-2 ® |
| somatrem | Protropin ® |
| dornase alfa | Pulmozyme ® |
| combination of ribavirin and alpha interferon | Rebetron ™ |
| Interferon beta 1a | Rebie |
| antihemophilic factor | Recombinate ® |
| rAHF/ntihemophilic factor | ReFacto ® |
| lepirudin | Refludan ® |
| reteplase | Retavase ™ |
| interferon alfa-2a | Roferon-A ® |
| somatropin | Saizen ® |
| synthetic porcine secretin | SecreFlo ™ |
| pegvisomant | Somavert ® |
| thyrotropin alfa | Thyrogen ® |
| tenecteplase | TNKase ™ |
| human immune globulin intravenous 5% and | Venoglobulin-S ® |
| 10% solutions | |
| interferon alfa-n1, lymphoblastoid | Wellferon ® |
| drotrecogin alfa | Xigris ™ |
| Somatotropin | Zorbtive ™(Serostim ®) |

Any of the antibodies or other products described above, can be a target protein for the methods described herein.

Exemplary Target and/or Test Protein Parameters

As used herein, a parameter associated with a test biologic, e.g., protein, refers to a characteristic of a test biologic, e.g., a moiety associated with the test biologic. In embodiments, the moiety is part of the test protein, e.g., connected with the rest of the test protein by a covalent bond, and the parameter is referred to herein as an intrinsic parameter. Intrinsic parameters include the presence, absence, level, ratio (with another entity), or distribution of a physical moiety, e.g., a moiety arising from or associated with a post-translational event. Exemplary parameters of this type include the presence, absence, level, ratio (with another entity), or distribution of a glycoform discussed herein.

Heavy Chain and Light Chain Amino Acid Sequence

An antibody can be described by its primary amino acid sequence. The chains are transcribed and translated from two independent genes and then assembled in the cell. Portions of the sequence are highly conserved across antibodies of the same class and species. For example, the Fc portion of the heavy chain is conserved across virtually human IgG1 antibodies. In contrast, the variable domains in the Fab portion of the heavy and light chains are unique to each antibody. Various methods can be used to determine the amino acid sequence, e.g., of the test protein and/or target protein. For example, peptide mapping can be used used with multiple enzymes to generate overlapping peptides that span the entire sequence.

C and N Termini

Antibodies commonly have modifications or truncations or extensions to their C or N termini. The carboxy termini of the IgG1 heavy chain, for example, terminates with a lysine moiety. These lysines can be enzymatically removed through the action of, e.g., a carboxypeptidase. Carboxypeptidase can be found in cell culture media, and may be released by lysed cells. CHO-expressed antibodies may have the lysines clipped off of one or both of their heavy chains. For efficient secretion of an antibody, the original gene construct requires an N terminal leader peptide for both the light and heavy chains. This peptide directs the translated peptide to the endoplasmic reticulum and onto the secretory pathway. Prior to secretion, the leader peptide is cleaved. Often, in particular with highly expressed proteins such as recombinant antibodies in CHO cells, a miscleavage of the leader peptide can occurs. This results in an additional amino acid or amino acids from the leader peptide on the antibody as it is secreted into the culture media.

Backbone Modification: Deamidation/Succinimide/isoAsp

Deamidation of asparagine residues is a commonly occurring post-translational modification (PTM) in antibodies and other biologic products. At neutral pH, Asn residues can cyclize as succinimide intermediates, with irreversible loss of NH3. This cyclic intermediate, while sometimes observed, is usually opened into either aspartic acid or iso-aspartic acid. All three of these Asn-derived PTMs (succinimide, isoAsp, and Asp) are classified as deamidations, and can be resolved, e.g., through a combination of chromatographic and mass spectrometric methods. Succinimide formation and Asp/isoAsp formation can be resolved, for example, as 17 Da and 1 Da mass changes, respectively, from the unmodified form, while the discrimination between Asp and isoAsp can be obtained from the chromatographic profiles of the deamidated peptides. Deamidation has been proposed to have an impact in multiple biological roles, including aging, amyloid diseases and activity of antibodies. Furthermore, deamidation is also used as a stability indicator. These modifications can be utilized to evaluate, e.g., the downstream process and formulation.

Backbone Modification: Pyroglutamate (pyroGlu)

When glutamine is present as the N-terminal amino acid on a protein, there is a potential for this residue to cyclize and form a pyroglutamate (pyroGlu) PTM. This reaction can also occur with N-terminal glutamic acid, but at a much lower rate of occurrence. This modification has been found in several proteins, including monoclonal antibodies and tends to increase upon extended storage, making it a useful stability indicator. As such these can be utilized, e.g., to evaluate downstream process and formulation.

Backbone Modification: Oxidation

Oxidation of backbone sidechains is another PTM found in proteins. While oxidation can occur on up to five different amino acids (His, Met, Tyr, Trp, and Cys), it is most commonly observed on Met and Cys residues. These residues can be oxidized by either O₂ or other reactive oxygen species, and the reaction can be significantly catalyzed by the presence of stray metal ions in solution. In monoclonal antibodies (mAbs), Met oxidation has been reported in both the Fab and Fc regions and can have a large spectrum of biological consequences, including reduction of activity, increased aggregation, and increased immunogenicity. Furthermore, suboptimal sample preparation conditions may lead to spurious Met oxidation.

While not typically present in mAbs (due to near-complete inclusion of available Cys residues into disulfide bonds), Cys oxidation can occur in other proteins, and has been shown to alter the higher-order structure of commercially produced cytokines.

Backbone Modification: Glycation

When in the presence of a reducing sugar such as sucrose, the amino group on lysine sidechains can become covalently linked to the exogenous saccharide through formation of a Schiff base. For proteins, this glycation PTM can occur either during the fermentation process, e.g., from sugars in the growth media, or it can occur post-purification if reducing sugars are present in the DP formulation.

Glycosylation

Glycosylation is the targeted attachment of oligosaccharides to specific amino acids. For the vast majority of antibodies, glycosylation occurs at a single glycosylation site in the Fc domain on the heavy chain, while others contain an additional one or two glycosylation sites in the Fab domain on either the heavy or the light chain. As the intact antibody is made of up two heavy chains and two light chains, the glycosylation is also redundant (e.g., for an antibody with only one glycosylation site on the heavy chain in the Fc portion, the intact antibody will often contain two oligosaccharides). In general, N-linked glycosylation is derived through a sequential series of sugar additions or removals resulting in structures that can contain, e.g., between 5 and 20 sugar moieties. The predominant sugar types include galactose, N-acetylglucosamine, N-acetylgalactosamine, mannose, fucose, and sialic acid. Some of these can be further modified to contain acetyl groups or additional sulfate moieties. The combination of variations in chain length, number of sugar building blocks, and the potential for modification is the reason N-linked glycosylation is the most diverse backbone modification. protein preparation can have hundreds of different glycan structures. In the case of antibodies, this is somewhat lessened by the position in the molecule, as the N-glycan site is internal to the molecule and is sterically hindered such that the general diversity of oligosaccharides is somewhat reduced. To this end, for most antibodies, the Fc glycosylation sites contain primarily biantennary glycans, with little to no sialylation. With that being said, the diversity within biantennary glycans is maintained, including high mannose variants as well as hybrid species and isomeric species.

Disulfide Linkages

Associations of amino acids of similar chemical characteristics lead to folding or turns in the linear peptide sequence to form macromolecular structural characteristics. A key example of these amino acid associations is the formation of disulfide linkages. In the appropriate reducing environment of the secretory pathway, cysteine moieties that come in close proximity to each other often form disulfide linkages. These connections may fold the molecule into unique confirmations or stabilize existing ones. For an antibody, these may be evaluated on three main levels, as indicated below.

Intrachain Disulfide Linkages

There are intrachain disulfide linkages in both the heavy and the light chain portions of the antibody. In the heavy chain, there are four disulfide linkages whereas with the light chain there are two. Although these typically happen between specific cysteine residues, in some cases, the cellular machinery can generate alternative connections that may impact the secondary structure of the antibody.

Heavy Chain, Light Chain Interchain Disulfide Linkages

The heavy chain and light chain are expressed as independent transcripts. For the formation of the intact antibody these chains are connected through a disulfide linkage as they move through the secretory pathway. For each heavy chain/light chain combination there is one disulfide linkage. Without appropriate interchain disulfide linkages free light chain or free heavy chain are secreted. To assure high expression, the light chain is often overexpressed leading to an excess of free light chain. To compensate for this, the light chain can be removed during the purification steps.

Heavy Chain, Heavy Chain Interchain Disulfide Linkages

The final heteromeric antibody requires the combination of the two heavy chain moieties. This relies on the formation of two disulfide linkages in the hinge region of the molecule. Similar to the other disulfide linkages, this occurs as the molecule moves through the secretory pathway. The combination of these two heavy chains forms the Fc portion of the antibody that is critical for effector functions such as ADCC or CDC. Furthermore, alternative disulfide linkages may impact the size of the pocket that will contain the glycan species. As such, the steric hindrance imparted through this domain may be removed and glycan composition may change significantly.

Characterization of the intrachain and interchain disulfide linkages can be used to define the higher order structure of the antibody molecule. Subtle differences in the disulfide connectivity have the potential to impact higher order structure that may not be captured using traditional structural analytics.

Higher Order Structure

Proteins such as antibodies have higher order structure beyond the amino acid sequence. Associations can occur between similarly charged amino acids and the molecule folds into a non-linear structure. Along the way, additional chemical linkages may occur (e.g., disulfide) to stabilize the confirmation.

In some embodiments, the higher order structure can be evaluated. For example, the secondary structure of a biologic can be evaluated. In some embodiments this may include, but not be limited to, evaluation of the extent of alpha-helical or beta-pleated sheet structures on a biologic. In other embodiments the amount of heavy chain:heavy chain dimers (HC:HC) or the levels of light chain:light chain dimers (LC:LC) can be evaluated. In other embodiments correlations between modifications can be evaluated (e.g. a correlation between the terminal lysine content on HC and the total glycan content).

Exemplary intrinsic parameters include: high mannose (e.g., HM3, HM5, HM6, HM7, HM8, and HM9); complex glycan (e.g., +/− fucosylation, and/or +/− sialylation, and/or +/− sulfation and the number of branches, e.g., biantennary, triantennary and tetraantennary); hybrid glycan; bisecting glycan; free cysteine (e.g., site-specific free cysteine, including global (e.g., total) free cysteine); disulfide connectivity A-B, where A and B are specific disulfides; pyroglutamate (e.g., N-terminal pyroglutamate, e.g., for antibodies, heavy chain and/or light chain N-terminal pyroglutamate); oxidation post-translational modifications (e.g., site specific oxidation post-translational modifications); succinimide post-translational modification; isoaspartic acid post-translational modification; glycation post-translational modification; C-terminal lysine (e.g., heavy and/or light chain); C-terminal amidation (e.g., heavy and/or light chain); and N-terminal fragmentation (e.g., heavy and/or light chain).

Other intrinsic parameters include, e.g., higher-order structure such as secondary structure (e.g., % alpha helix content; % beta sheet content); tertiary structure (e.g., extent of protein folding as measured by intrinsic fluorescence or ANS dye fluorescence); tertiary structure and dynamics (e.g., accessibility of amide protons to solvent water as measured by hydrogen-deuterium exchange); and % aggregation, e.g., monitored by either SEC or analytical ultracentrifugation.

In some instances, test and/or target protein parameters, including determinative parameters, can include, but are not limited to, one or more, at least one, a plurality, or all of the parameters listed in Table 2.

TABLE 2

| | | Parameter |
|---|---|---|
| Parameter # | Parameter Category | 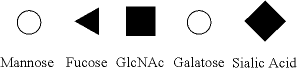 |
| 1 | HM3 | 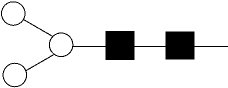 |
| 2 | HM4 | 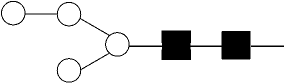 |
| 3 | HM5 | 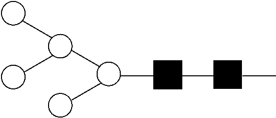 |
| 4 | HM6 | 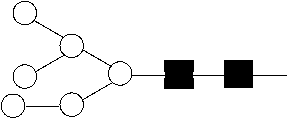 |
| 5 | HM7 | 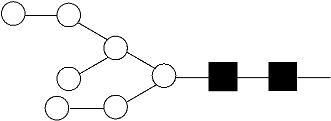 |
| 6 | HM8 | 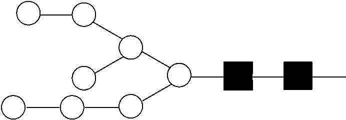 |
| 7 | HM9 | 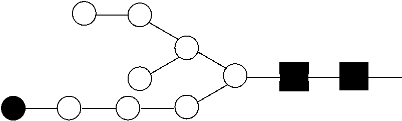 |
| 8 | Complex | 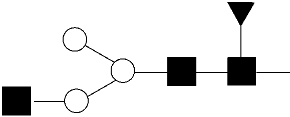 |
| 9 | Complex | 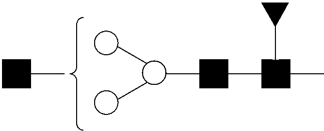 |
| 10 | Complex | 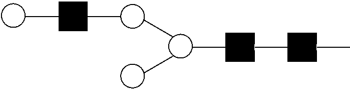 |
| 11 | Complex | 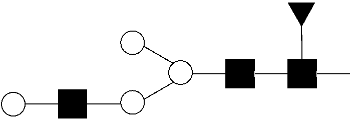 |
| 12 | Complex G0 | 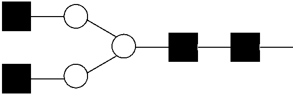 |
| 13 | Complex G0F | 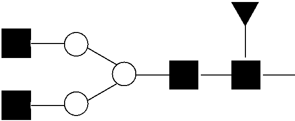 |
| 14 | Complex G1 | 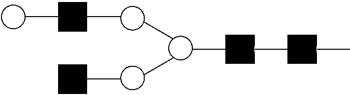 |
| 15 | Complex G1 | 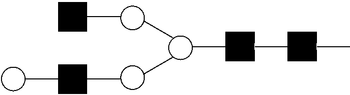 |
| 16 | Complex G1F | 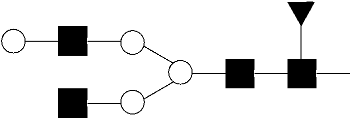 |
| 17 | Complex G1F | 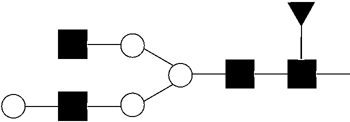 |
| 18 | Complex G2 | 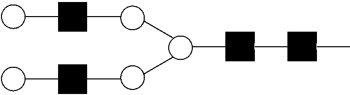 |
| 19 | Complex G2F | 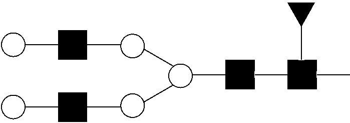 |
| 20 | Hybrid | 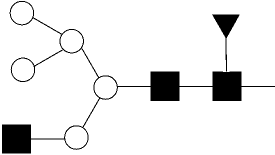 |
| 21 | Hybrid | 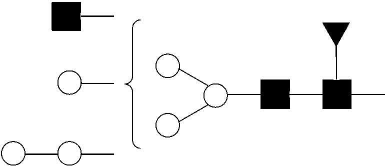 |
| 22 | Hybrid | 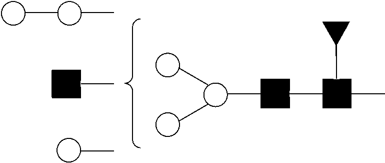 |
| 23 | Bisecting | 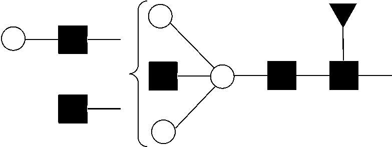 |
| 24 | Bisecting | 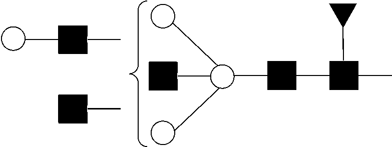 |
| 25 | Sialylated | 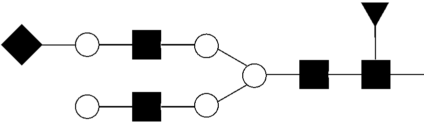 |
| 26 | Sialylated | 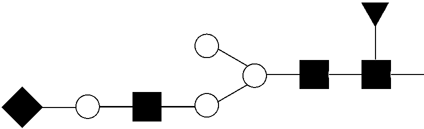 |
| 27 | Sialylated | 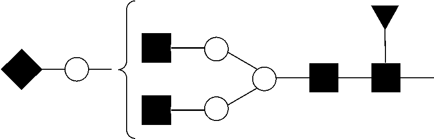 |
| 28 | Sialylated | 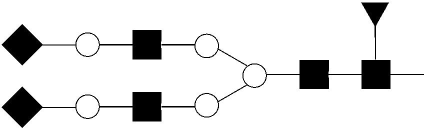 |
| 29 | C-Terminal- | Amount of lysine present at the C-terminus of the |
| | lysine | heavy chain |
| 30 | HC-Pyroglu | Pyroglutamate (pyroglu) at the N-terminus of the |
| | | heavy chain |
| 31 | LC-Pyroglu | Pyroglutamate at the N-terminus of the light |
| | | chain |
| 32 | HC-M256- | Post-translational modification of the M256 |
| | Sulfo | residue (Kabat et al. numbering) of the heavy |
| | | chain-residue is oxidized to form methionine |
| | | sulfoxide |
| 33 | LC-K149-Glyc | Post-translational glycation at lysine 149 of the |
| | | light chain |
| 34 | LC-135 | Amount of free cysteine (e.g. not paired in |
| | | disulfides) at cysteine 135 in the light chain |
| 35 | LC-D17-Suc | Succinimide formation at aspartic acid 17 on the |
| | | light chain |
| 36 | HC148 | Amount of free cysteine (e.g. not paired in |
| | | disulfides) at cysteine 148 in the heavy chain |
| 37 | HC204 | Amount of free cysteine (e.g. not paired in |
| | | disulfides) at cysteine 204 in the heavy chain |
| 38 | HC265 | Amount of free cysteine (e.g. not paired in |
| | | disulfides) at cysteine 265 in the heavy chain |
| 39 | HC371 | Amount of free cysteine (i.e., not paired in |
| | | disulfides) at cysteine 371 in the heavy chain |
| 40 | HC429 | Amount of free cysteine (i.e. not paired in |
| | | disulfides) at cysteine 429 in the heavy chain |

For related parameters with the same listed structure (e.g., parameter numbers 8 and 9; 14 and 15; 16 and 17; 20, 21, and 22; 23 and 24; and 26 and 27) the listed isomers are assigned in order of their retention time from a reverse-phase C18 column.

In some instances, parameters, including those provided in Table 2, include one or more high mannose glycans, one or more complex glycans, one or more hybrid glycans, one or more sialylated glycans, bisecting glycans (e.g., bisecting glycan A and/or B), and combinations thereof.

In other embodiments, the moiety is not part of the test protein but is present in the sample with the test protein and the parameter is referred to herein as a sample, or extrinsic, parameter. Exemplary parameters of this type include the presence, absence, level, ratio (with another entity), or distribution of impurities, e.g., whole cell proteins, residue from purification processes, viral contaminants, and enclosure components.

### Parameter Evaluation

Parameters disclosed herein can be analyzed by any available suitable method. In some instances, glycan structure and composition as described herein are analyzed, for example, by one or more, enzymatic, chromatographic, mass spectrometry (MS), chromatographic followed by MS, electrophoretic methods, electrophoretic methods followed by MS, nuclear magnetic resonance (NMR) methods, and combinations thereof. Exemplary enzymatic methods include contacting a glycoprotein preparation with one or more enzymes under conditions and for a time sufficient to release one or more glycan(s) (e.g., one or more exposed glycan(s)). In some instances, the one or more enzymes include(s) PNGase F. Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some instances, techniques described herein may be combined with one or more other technologies for the detection, analysis, and or isolation of glycans or glycoproteins. For example, in certain instances, glycans are analyzed in accordance with the present disclosure using one or more available methods (to give but a few examples, see Anumula, Anal. Biochem., 350(1):1, 2006; Klein et al., Anal. Biochem., 179:162, 1989; and/or Townsend, R. R. Carbohydrate Analysis” High Performance Liquid Chromatography and Capillary Electrophoresis., Ed. Z. El Rassi, pp 181-209, 1995; WO2008/128216; WO2008/128220; WO2008/128218; WO2008/130926; WO2008/128225; WO2008/130924; WO2008/128221; WO2008/128219; WO2008/128222; WO2010/071817; WO2010/071824; WO2010/085251; WO2011/069056; and WO2011/127322, each of which is incorporated herein by reference in its entirety). For example, in some instances, glycans are characterized using one or more of chromatographic methods, electrophoretic methods, nuclear magnetic resonance methods, and combinations thereof.

In some instances, methods for evaluating one or more target protein specific parameters, e.g., in a glycoprotein preparation, e.g., one or more of the parameters disclosed herein, can be performed by one or more of following methods.

TABLE 3
Exemplary Methods of evaluating parameters

| Method(s) | Relevant literature | Parameter |
|---|---|---|
| C18 UPLC Mass Spec.* | Chen and Flynn, Anal. Biochem., | Glycan(s) |
| | 370: 147-161 (2007) | (e.g., N-linked glycan, exposed N- |
| | Chen and Flynn, J. Am. Soc. Mass | linked glycan, glycan detection, |
| | Spectrom., 20: 1821-1833 (2009) | glycan identification, and |
| | | characterization; site specific |
| | | glycation; glycoform detection; |
| | | percent glycosylation; and/or |
| | | aglycoosyl) |
| Peptide LC-MS | Dick et al., Biotechnol. Bioeng., | C-terminal lysine |
| (reducing/non-reducing) | 100: 1132-1143 (2008) | |
| | Yan et al., J. Chrom. A., 1164: 153-161 | |
| | (2007) | |
| | Chelius et al., Anal. Chem., 78: 2370- | |
| | 2376 (2006) | |
| | Miller et al., J. Pharm. Sci., 100: 2543- | |
| | 2550 (2011) | |
| LC-MS (reducing/non- | Dick et al., Biotechnol. Bioeng., | |
| reducing/alkylated) | 100: 1132-1143 (2008) | |
| | Goetze et al., Glycobiol., 21: 949-959 | |
| | (2011) | |
| Weak cation exchange | Dick et al., Biotechnol. Bioeng., | |
| (WCX) chromatography | 100: 1132-1143 (2008) | |
| LC-MS (reducing/non- | Dick et al., Biotechnol. Bioeng., | |
| reducing/alkylated) | 100: 1132-1143 (2008) | |
| | Goetze et al., Glycobiol., 21: 949-959 | |
| | (2011) | |
| PeptideLC-MS | Yan et al., J. Chrom. A., 1164: 153-161 | N-terminal pyroglu |
| (reducing/non-reducing) | (2007) | |
| | Chelius et al., Anal. Chem., 78: 2370- | |
| | 2376 (2006) | |
| | Miller et al., J. Pharm. Sci., 100: 2543- | |
| | 2550 (2011) | |
| Peptide LC-MS | Yan et al., J. Chrom. A., 1164: 153-161 | Methionine oxidation |
| (reducing/non-reducing) | (2007); | |
| | Xie et al., mAbs, 2: 379-394 (2010) | |
| Peptide LC-MS | Miller et al., J. Pharm. Sci., 100: 2543- | Site specific glycation |
| (reducing/non-reducing) | 2550 (2011) | |
| Peptide LC-MS | Wang et al., Anal. Chem., 83: 3133-3140 | Free cysteine |
| (reducing/non-reducing) | (2011); | |
| | Chumsae et al., Anal. Chem., 81: 6449- | |
| | 6457 (2009) | |
| Bioanalyzer | Forrer et al., Anal. Biochem., 334: 81-88 | Glycan (e.g., N-linked glycan, |
| (reducing/non-reducing)* | (2004) | exposed N-linked glycan) |
| | | (including, for example, glycan |
| | | detection, identification, and |
| | | characterization; site specific |
| | | glycation; glycoform detection; |
| | | percent glycosylation; and/or |
| | | aglycoosyl) |
| LC-MS (reducing/non- | Dick et al., Biotechnol. Bioeng., | Glycan (e.g., N-linked glycan, |
| reducing/alkylated)* | 100: 1132-1143 (2008) | exposed N-linked glycan) |
| * Methods include | Goetze et al., Glycobiol., 21: 949-959 | (including, for example, glycan |
| removal (e.g., enzymatic, | (2011) | detection, identification, and |
| chemical, and physical) | Xie et al., mAbs, 2: 379-394 (2010) | characterization; site specific |
| of glycans | | glycation; glycoform detection; |
| | | percent glycosylation; and/or |
| | | aglycoosyl) |
| Bioanalyzer | Forrer et al., Anal. Biochem., 334: 81-88 | Light chain: Heavy chain |
| (reducing/non-reducing) | (2004) | |
| Peptide LC-MS | Yan et al., J. Chrom. A., 1164: 153-161 | Non-glycosylation-related peptide |
| (reducing/non-reducing) | (2007) | modifications (including, for |
| | Chelius et al., Anal. Chem., 78: 2370- | example, sequence analysis and |
| | 2376 (2006) | identification of sequence variants; |
| | Miller et al., J. Pharm. Sci., 100: 2543- | oxidation; succinimide; aspartic |
| | 2550 (2011) | acid; and/or site-specific aspartic |
| | | acid) |
| Weak cation exchange | Dick et al., Biotechnol. Bioeng., | Isoforms (including, for example, |
| (WCX) chromatography | 100: 1132-1143 (2008) | charge variants (acidic variants |
| | | and basic variants); and/or |
| | | deamidated variants) |
| Anion-exchange | Ahn et al., J. Chrom. B, 878: 403-408 | Sialylated glycan |
| chromatography | (2010) | |
| Anion-exchange | Ahn et al., J. Chrom. B, 878: 403-408 | Sulfated glycan |
| chromatography | (2010) | |
| 1,2-diamino-4,5- | Hokke et al., FEBS Lett., 275: 9-14 | Sialic acid |
| methylenedioxybenzene | (1990) | |
| (DMB) labeling method | | |
| LC-MS | Johnson et al., Anal. Biochem., 360: 75- | C-terminal amidation |
| | 83 (2007) | |
| LC-MS | Johnson et al., Anal. Biochem., 360: 75- | N-terminal fragmentation |
| | 83 (2007) | |
| Circular dichroism | Ham et al., Current Trends in | Secondary structure (including, for |
| spectroscopy | Monoclonal Antibody Development and | example, alpha helix content |
| | Manufacturing, S. J. Shire et al., eds, | and/or beta sheet content) |
| | 229-246 (2010) | |
| Intrinsic and/or ANS dye | Ham et al., Current Trends in | Tertiary structure (including, for |
| fluorescence | Monoclonal Antibody Development and | example, extent of protein folding) |
| | Manufacturing, S. J. Shire et al., eds, | |
| | 229-246 (2010) | |
| Hydrogen-deuterium | Houde et al., Anal. Chem., 81: 2644- | Tertiary structure and dynamics |
| exchange-MS | 2651 (2009) | (including, for example, |
| | | accessibility f amide protons to |
| | | solvent water) |
| Size-exclusion | Carpenter et al., J. Pharm. Sci., 99: 2200- | Extent of aggregation |
| chromatography | 2208 (2010) | |
| Analytical | Pekar and Sukumar, Anal. Biochem., | |
| ultracentrifugation | 367: 225-237 (2007) | |

The literature recited above are hereby incorporated by reference in their entirety or, in the alternative, to the extent that they pertain to one or more of the methods for determining a parameter described herein.

### Determinative and Non-Determinative Test Protein Parameters

The methods described herein include, inter alia, processing a test protein preparation if the input values for one or a plurality of determinative test protein parameters of the test protein meet a preselected criteria of target protein values for such parameters. In addition, the methods described herein can also include determining or acquiring values for non-determinative test protein parameters for the test protein preparation.

An input value, e.g., an input value for a determinative parameter for a test protein is indistinguishable from a preselected criterion of target protein values for such parameter, e.g., the value for said determinative parameter for a target biologic, when the input value is within (e.g., is the same as or is within the range, limits or specifications for) the preselected criteria of a target. Likewise, an input value, e.g., an input value for a determinative parameter for a test protein meets a preselected criterion of target protein values for such parameter, e.g., the value for said determinative parameter for a target biologic, when the input value is within (e.g., is the same as or is within the range, limits or specifications for) the preselected criteria of a target. The range, limits or specifications for a target biologic's parameter's value may be determined, e.g., as the average value or range of values (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 25, 30, 40, 50 or more samples, e.g., commercially available samples or batches, of the target protein. In some embodiments, a preselected criterion is a release specification for a given parameter for release of the test protein as a 351(k) licensed product (a biosimilar or interchangeable product) that reflects the average value or range of values for the parameter (e.g., a range including the minimum and maximum values, and in some cases plus or minus a window of variability (e.g., +/−10%, +/−15%, +/−20% or +/− one or two standard deviations) to account for analytical and/or sample variability in the target) for any 2, 3, 4, 5, 6, 7, 8, 9, 10, 15, 20, 25, 30, 40, 50 or more samples, e.g., commercially available samples or batches, of the target protein. In some embodiments, the input value can be an average value, e.g., a measure of central tendency. For example, an input value can reflect multiple datapoints (e.g., from multiple reads of a single sample and/or multiple samples of a single batch of a test protein) for a single test protein.

Information (e.g., input value(s) and/or preselected criteria) pertaining to a parameter means information, regardless of form, that describes the presence, absence, abundance, absolute or relative amount, ratio (with another entity), or distribution of a characteristic (e.g., a moiety) associated with a test biologic and/or a target biologic. Such information can be qualitative, e.g., present, absent, intermediate, or quantitative, e.g., a numerical value such as a single number, or a range, for a parameter. In some instances, information can be, for example: a statistical function, e.g., an average, of a number of values; a function of another value, e.g., of the presence, distribution or amount of a second entity present in the sample, e.g., an internal standard; a statistical function, e.g., an average, of a number of values; a function of another value, e.g., of the presence, distribution or amount of a second entity present in the sample, e.g., an internal standard; a value, e.g., a qualitative value, e.g., present, absent, “below limit of detection”, “within normal limits” or intermediate. In some instances, information can be a quantitative value, e.g., a numerical value such as a single number, a range of values, a “no less than x amount” value, a “no more than x amount” value. In some instances, information can be abundance. Abundance can be expressed in relative terms, e.g., abundance can be expressed in terms of the abundance of a structure in relation to another component in the preparation. E.g., abundance can be expressed as: the abundance of a structure (or a first group of structures) in Table 2A-E relative to the amount of protein; the abundance of a structure (or a first group of structures) in Table 2A-E relative to the abundance of a second structure (or second group of structures) in Table 2A-E. Abundance, e.g., abundance of a first structure relative to another structure, can be with regard to the preparation as a whole, a single molecule, or a selected site on the protein backbone. E.g., the parameter can be the relative proportion of a first structure from Table 2A-E and a second structure from Table 2A-E at a selected site and the value can be expressed as, e.g., a proportion, ratio or percentage. Information can be expressed in any useful term or unit, e.g., in terms of weight/weight, number/number, number/weight, and weight/number.

In some embodiments, a preselected criteria can be a signature, wherein the signature comprises a plurality of target protein values (e.g., for determinative and/or non-determinative parameters). A parameter (e.g., for a target protein) can be categorized as a determinative test protein parameter or non-determinative test protein parameter (or non-determinative entry) by the methods described herein. For example, for a target protein whether a parameter is determinative or non-determinative can be determined as follows: values for a plurality of parameters are determined for a plurality of lots or samples of a plurality of products (e.g., of multiple distinct therapeutic protein products). Parameters for which the values are consistently similar or non-distinguishing, e.g., invariant, across the plurality of products are discarded from consideration as determinative test protein parameters (e.g., for a particular member of the plurality). If a parameter for which a value associated with a single product is unique relative to that parameter's values in others of the plurality, the parameter is assigned a rule specifying the uniqueness. The rule may be, e.g., “present,” absent,” “greater than X,” “less than X,” or a “within a range of X-Y” for the parameter value for the relevant single product. This can be repeated for each parameter (generating a new rule each time) until no uniqueness for that particular product remains. The unique parameters for a protein as compared to others of the protein plurality are considered determinative protein parameters for that protein. In some embodiments, members of the plurality are members of the same class of proteins. For example, the plurality is all: antibodies, the same class of antibodies, the same isotype, Fc-containing proteins, CDR-grafted antibodies, humanized antibodies, or human antibodies.

In addition, a parameter can be considered a determinative test protein parameter if the value associated with a parameter for two or more proteins is unique for those two or more proteins relative to others of the plurality. Such parameters can be a determinative test protein parameter for each of the two or more proteins relative to the other proteins of the plurality for this parameter. However, this same parameter would be considered a non-determinative test protein parameter between the two or more such proteins.

A plurality of determinative test protein parameters (and non-determinative test protein parameters) can be compiled for any target protein.

In some embodiments, the level of similarity between a test protein and a target protein may be expressed as a sameness/identity, or s/i value. The s/i value is a function of the relationship between a plurality of input values for test protein parameters and a preselected or predefined plurality of target values for a target protein (e.g., a signature). For example, a high s/i value reflects a high level of similarity between a plurality of input values for test protein parameters and a preselected or predefined plurality of target values for a target protein (e.g., a signature). For example, a s/i of 1 may represent a plurality of input values for test protein parameters that is indistinguishable from a preselected or predefined plurality of target values for a target protein (e.g., a signature), whereas any s/i value less than 1, but greater than 0, signals that the plurality of input values and the preselected or predefined plurality of target values for a target protein (e.g., the signature) are not indistinguishable but have some level of similarity.

In some embodiments, where an s/i value is less than 1, analysis of indistinguishability and/or similarity can include consideration of difference (e.g., difference of parameter values between test and target) and, optionally, the relevance of such difference. In some embodiments, consideration includes comparison of seriousness values for the parameters. A seriousness value is a quantitative or qualitative value and is a function of a risk associated with variation in the parameter, e.g., a determinative test protein parameter. In embodiments, the risk is the risk of a difference from the target in the level of efficacy or safety, the risk of an unacceptable level of difference in efficacy or safety (e.g., below a predetermined standard) from the target protein. In embodiments, a seriousness value, or seriousness values for a plurality of parameters, is selected (e.g., by assigning the appropriate numerical values) such that consideration of additional parameters does not alter a determination or outcome with regard to the generated sameness/interchangeability value.

In some embodiments, a seriousness value can be provided for a parameter. For example, a parameter that has a high seriousness value can be a determinative test protein parameter in the methods described herein. In some embodiments, all of the determinative test protein parameters of the plurality have a seriousness value. The target protein value, e.g., a range, can be adjusted depending on the seriousness value for that parameter. For example, the seriousness value is given a numerical value from 0 to 100, with 0 being no risk, 100 being risk associated with a serious adverse event such as death, and a score between 1 and 99 signals an increasing level of risk with 1 equaling low risk and 99 equaling high risk. For example, a terminal galactose-alpha-1-3-galactose parameter may be assigned a high seriousness value.

In one embodiment, the plurality of determinative test biologic parameters includes parameters having a seriousness value of greater than 1, 10, 20, 30, 40, 50, 60, 70, 80, or 90. In one embodiment, all of the determinative test biologic parameters acquired or evaluated for parameters have a seriousness value of greater than 50, 60, 70, 80, or 90.

In some embodiments, s/i is a function of: parameters measured, distance between test and target values for parameters, and, optionally, seriousness value for parameters.

### Recombinant Gene Expression

A test protein preparation described herein can be produced, e.g., recombinantly, employing conventional molecular biology, microbiology, and recombinant DNA techniques within the skill of the art. Such techniques are described in the literature (see, e.g., Sambrook, Fritsch & Maniatis, Molecular Cloning: A Laboratory Manual, Third Edition (2001) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1995).

Techniques for sequencing a polypeptide to determine its amino acid sequence and for making polynucleotides that encode a particular desired amino acid sequence are routine in the art. Recombinant expression of a gene or cDNA, such as a gene or cDNA encoding a protein (e.g., antibody) described herein, can include construction of an expression vector containing a polynucleotide that encodes a desired protein or antibody. Once a polynucleotide has been obtained, a vector for the production of the encoded polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce a recombinant polypeptide. A variety of host expression vector systems can be used (see, e.g., U.S. Pat. No. 5,807,715). Such host-expression systems can be used to produce polypeptides. Such host expression systems include microorganisms such as bacteria (e.g., E. coli and B. subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g. Ti plasmid) containing polypeptide coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For expression in mammalian host cells, viral-based expression systems can be utilized (see, e.g., Logan & Shenk, 1984, Proc. Natl. Acad. Sci. USA 8 1:355-359). The efficiency of expression can be enhanced by the inclusion of appropriate transcription enhancer elements, transcription terminators, etc. (see, e.g., Bittner et al., 1987, Methods in Enzymol. 153:516-544).

In addition, a host cell strain can be chosen that modulates the expression of the inserted sequences, or modifies and processes the gene product in the specific fashion desired. Different host cells have characteristic and specific mechanisms for the post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, but are not limited to, CHO, VERY, BHK, HeLa, COS, MDCK, 293, 3T3, W138, BT483, Hs578T, HTB2, BT20 and T47D, NSO (a murine myeloma cell line that does not endogenously produce any immunoglobulin chains), CRL7O3O and HsS78Bst cells.

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a glycoprotein described herein been produced by recombinant expression, it may be purified by any method known in the art for purification, for example, by chromatography (e.g., ion exchange, affinity, and sizing column chromatography), centrifugation, differential solubility, or by any other standard technique for the purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a glycoprotein can be fused to heterologous polypeptide sequences to facilitate purification. Glycoproteins having desired sugar chains can be separated with a lectin column by methods known in the art (see, e.g., WO 02/30954).

### Pharmaceutical Compositions and Administration

A protein (e.g., antibody) preparation described herein can be incorporated into a pharmaceutical composition. A protein preparation may be formulated for pharmaceutical use by methods known to those skilled in the art. The pharmaceutical composition can be administered parenterally in the form of an injectable formulation comprising a sterile solution or suspension in water or another pharmaceutically acceptable liquid. For example, the pharmaceutical composition can be formulated by suitably combining the produced, purified protein with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection. A suitable means of administration can be selected based on the age and condition of the patient. A single dose of the pharmaceutical composition containing a sulfated glycoprotein can be selected from a range of 0.001 to 1000 mg/kg of body weight. On the other hand, a dose can be selected in the range of 0.001 to 100000 mg/body weight, but the present disclosure is not limited to such ranges. The dose and method of administration varies depending on the weight, age, condition, and the like of the patient, and can be suitably selected as needed by those skilled in the art.

### EXAMPLES

### Example 1

### Target Protein Characterization

Characterization of target protein X (a monoclonal antibody) was performed by orthogonal methods. Measurements were made as described herein and included glycan profiling, glycoform analysis, post-translational modification analysis, and analysis of other intrinsic and extrinsic structures or features of target protein X. Of 113 structures or features that were measured or determined, 10 were determined to be determinative parameters for target protein X. Values for these 10 target protein X parameters are listed in Table 4 below.

TABLE 4
Values for Target Protein A Determinative Parameters

| | | Structure or description | |
|---|---|---|---|
| Parameter # | Parameter Class | 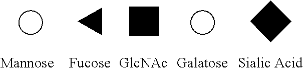 | Value |
| 1 | Complex G0F | 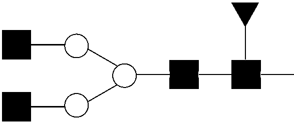 | 56.96 |
| 2 | Complex | 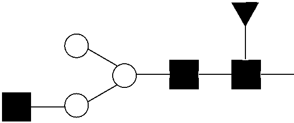 | 1.78 |
| 3 | Complex G2F | 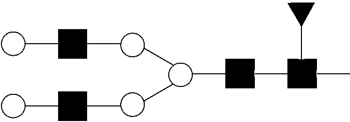 | 4.07 |
| 4 | Complex G0 | 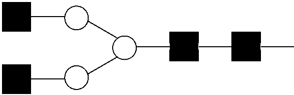 | 0.31 |
| 5 | Complex G1 | 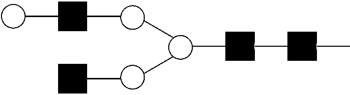 | 0.05 |
| 6 | Complex G1 | 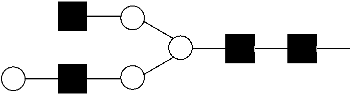 | 0.01 |
| 7 | C-terminal- | Amount of lysine present at the C-terminus of the | 19.40 |
| | lysine | heavy chain | |
| 8 | HC-pyroglu | Pyroglutamate at the N-terminus of the heavy | 100.00 |
| | | chain | |
| 9 | LC-pyroglu | Pyroglutamate at the N-terminus of the light chain | 88.30 |
| 10 | LC135 | Amount of free cysteine (i.e., not paired in | 3.30 |
| | | disulfides) at cysteine 135 in the light chain | |

The information (values) shown for each determinative target protein X parameter in Table 4 above were used to formulate a reference criterion or rule for each determinative target protein X parameter. These rules are shown in Table 5 below.

TABLE 5

| | | Structure or description | |
|---|---|---|---|
| Parameter # | Parameter Class | 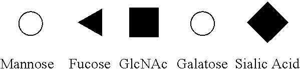 | Reference Criterion (rule) |
| 1 | Complex G0F | 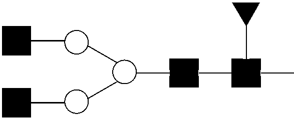 | >45.00%* |
| 2 | Complex | 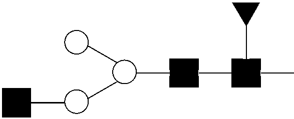 | >0.60%* |
| 3 | Complex G2F | 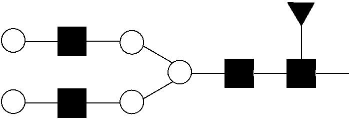 | >3.50%* |
| 4 | Complex G0 | 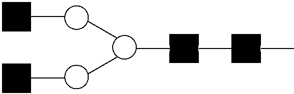 | <0.90%* |
| 5 | Complex G1 | 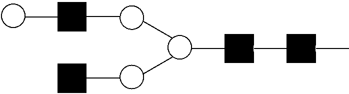 | <0.10%* |
| 6 | Complex G1 | 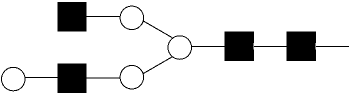 | <0.05%* |
| 7 | C-terminal- | Amount of lysine present at the C-terminus of the | <25.00%^{$} |
| | lysine | heavy chain | |
| 8 | HC-pyroglu | Pyroglutamate at the N-terminus of the heavy | >80.00%^{#} |
| | | chain | |
| 9 | LC-pyroglu | Pyroglutamate at the N-terminus of the light chain | >60.00%^{#} |
| 10 | LC135 | Amount of free cysteine (i.e., not paired in | >2.00%\hat{ } |
| | | disulfides) at cysteine 135 in the light chain | |

*For any given parameter, percent refers to the number of moles of PNGase F-released glycan X relative to total moles of PNGase F-released glycan detected as disclosed in Table 3, wherein X represents the parameter of interest (e.g., parameter(s) 1-11).
^{#}For any given parameter, percent refers to the level of modified peptide Y relative to the sum of the levels of modified peptide Y and unmodified peptide Y, detected as disclosed in Table 3, wherein Y represents the parameter of interest (e.g., parameter(s) 13, 14).
^{$}For C-terminal-lysine, percent refers to the level of C-terminal-lysine-containing peptide relative to the sum of the levels of C-terminal-lysine-containing and C-terminal-lysine-free peptides detected as disclosed in Table 3.
\hat{ }For free cysteine, percent refers to the level of non-disulfide-linked peptide relative to the sum of the levels of non-disulfide-linked and disulfide-linked peptides, detected as disclosed in Table 3.

### Example 2

### Evaluation of Test Protein Preparations

The reference criteria or rules (the predefined plurality of target values) in Example 1 were used to determine whether test proteins (test proteins 1 and 2) are similar or identical to target protein X. Test proteins 1 and 2 are two different monoclonal antibodies.

Test protein 1 was analyzed and input values were obtained for each of the determinative target protein X parameters. The values of these parameters for test protein 1 are presented in Table 6 below. Values obtained for test protein 1 were compared to the reference criteria or rules (the predefined plurality of target values for target protein X), shown in Table 6 below:

TABLE 6

| | | Target | |
|---|---|---|---|
| | Test Protein 1 | Protein A | Test Versus |
| Parameter # | value | Rule | Target |
| 1 | 68.46 | >45.00 | ✓ |
| 2 | 1.28 | >0.60 | ✓ |
| 3 | 2.26 | >3.50 | |
| 4 | 2.17 | <0.90 | |
| 5 | 0.26 | <0.10 | |
| 6 | 0.13 | <0.05 | |
| 7 | 1.60 | <25.00 | ✓ |
| 8 | 2.30 | >80.00 | |
| 9 | 0.00 | >60.00 | |
| 10 | 0.70 | >2.00 | |

✓ Illustrates that a value meets the reference criterion/rule.

As shown above, in an exemplary plurality of 10 determinative target protein parameters for protein X, only two test protein input values were indistinguishable from the corresponding determinative target protein X parameters. This suggests a low s/i value and/or that test protein 1 is not sufficiently similar to target protein X to be processed as a pharmaceutical product equivalent to target protein X.

Test protein 2 was also analyzed and values were obtained for each of the determinative target protein X parameters. The values of these parameters for test protein 2 are presented in Table 7 below. Values obtained for test protein 2 were compared to the reference criteria or rules (the predefined plurality of target values for target protein X), shown in Table 7 below:

TABLE 7

| | | Target | |
|---|---|---|---|
| | Test Protein 2 | Protein A | Test Versus |
| Parameter # | value | Rule | Target |
| 1 | 56.96 | >45.00 | ✓ |
| 2 | 1.78 | >0.60 | ✓ |
| 3 | 4.07 | >3.50 | ✓ |
| 4 | 0.31 | <0.90 | ✓ |
| 5 | 0.05 | <0.10 | ✓ |
| 6 | 0.01 | <0.05 | ✓ |
| 7 | 19.40 | <25.00 | ✓ |
| 8 | 100.00 | >80.00 | ✓ |
| 9 | 88.30 | >60.00 | ✓ |
| 10 | 3.30 | >2.00 | ✓ |

✓ Illustrates that a value meets the reference criterion/rule.

As shown above, all determinative parameters of the plurality of 10 were indistinguishable between test protein 2 and target protein X. Thus, target protein 2 has a high s/i value and may be processed as a pharmaceutical drug product equivalent to and/or interchangeable with target protein X.

### Example 3

### Evaluation of Test Protein 3

Test protein 3 is marketed as a target protein X “biosimilar” in certain non-US jurisdictions. Test protein 3 is a monoclonal antibody directed against the same antigen as target protein X and shares 100% amino acid sequence identity to target protein X. Test protein 3 was analyzed and values were obtained for each of the determinative target protein X parameters. The values of these parameters for test protein 3 are presented in Table 8 below. Values obtained for test protein 3 were compared to the reference criteria or rules (the predefined plurality of target values), shown in Table 8 below:

TABLE 8

| | | Target | |
|---|---|---|---|
| | Test Protein 3 | Protein A | Test Versus |
| Parameter # | value | Rule | Target |
| 1 | 48.30 | >45.00 | ✓ |
| 2 | 1.03 | >0.60 | |
| 3 | 2.96 | >3.50 | |
| 4 | 3.56 | <0.90 | |
| 5 | 0.81 | <0.10 | |
| 6 | 0.31 | <0.05 | |
| 7 | 46.90 | <25.00 | |
| 8 | 100.00 | >80.00 | ✓ |
| 9 | 75.40 | >60.00 | ✓ |
| 10 | 1.60 | >2.00 | |

✓ Illustrates that a value meets the reference criterion/rule.

As shown above, in the exemplary plurality of 10 determinative target protein parameters for protein X, only three test protein 3 input values were indistinguishable from the corresponding determinative target protein X parameters. This suggests that although target protein X and test protein 3 are identical in amino acid sequence and directed against the same antigen, test protein 3 is not sufficiently similar to target protein X to be processed as a pharmaceutical product equivalent to target protein X according to the methods described herein, even if the plurality consisted only of 4 determinative test biologic parameters.

While the methods have been described in conjunction with various instances and examples, it is not intended that the methods be limited to such instances or examples. On the contrary, the methods encompass various alternatives, modifications, and equivalents, as will be appreciated by those of skill in the art.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

1.-114. (canceled)

115. A method of manufacturing a pharmaceutical product comprising a protein, the method comprising:
producing a test protein preparation, wherein the test protein is not approved under a biologics license application (BLA) or a supplemental BLA;
obtaining a signature for the test protein, wherein the signature comprises a plurality, e.g., at least 2, of values for determinative test protein parameters that distinguish the test protein from a plurality of non-test proteins; and
processing the test protein preparation into a pharmaceutical product if the signature for the test protein is indistinguishable from a predetermined signature (of the determinative test protein parameters) for a target protein, wherein the target protein has an amino acid sequence that is substantially the same as the test protein amino acid sequence (e.g., the target protein has an amino acid sequence that is at least 95%, 96%, 97%, 98%, 99%, or 100% identical to the test protein amino acid sequence or which differs by less than 10, 5, 4, 3 or less amino acids from the test protein amino acid sequence), and wherein the target protein is approved under a BLA, a supplemental BLA, or an equivalent thereof,
thereby manufacturing a pharmaceutical product comprising a protein.

116. (canceled)

117. The method of claim 115, wherein at least one input value, e.g., a value of a determinative test protein parameter, is directly acquired or is acquired by performing an analytical analysis on said test protein preparation.

118.-120. (canceled)

121. The method of claim 115, wherein processing or formulating comprises combining the test protein preparation with a second component, e.g., an excipient or buffer.

122. The method of claim 115, wherein processing or formulating comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label; shipping or moving the test protein preparation to a different location.

123. The method of claim 115, wherein each of the values for the plurality of determinative test protein parameters is indistinguishable from its corresponding target protein value.

124. The method of claim 115, wherein a determinative test protein parameter is indistinguishable from the value for that parameter (individually) in any 1, 2, 3, 4, 5, 6, 7, 8, 9, or 10, or more, commercially available samples, or batches, of the target protein or is indistinguishable from the average value (or other measure of central tendency), or falls within the range for the value, for any 2, 3, 4, 5, 6, 7, 8, 9, or 10, or more, commercially available samples, or batches, of the target protein.

125. (canceled)

126. The method of claim 115, further comprising, providing the average value (or other measure of central tendency) or range of values for a parameter for 2, 3, 4, 5, 6, 7, 8, 9, or 10, or more, samples or batches of the target protein and comparing it with the value for the determinative test protein parameter from the test protein.

127. The method of claim 115, wherein the plurality of determinative test biologic parameters includes at least 4 (5, 6, 7, 8, 9, 10, or more) determinative test biologic parameters.

128. The method of claim 115, wherein the value for the test protein preparation is from one sample or batch of test protein.

129. The method of claim 115, wherein the value, e.g., an average value or range of values, for the test protein is derived from 2, 3, 4, 5, 6, 7, 8, 9, or 10, or more, samples or batches of test protein.

130. The method of claim 115, wherein a value for a determinative test protein parameter is indistinguishable from, or falls within, the target protein value, if the value of the determinative test protein parameter is a release specification for that parameter that reflects the average range of values (including the minimum and maximum values +/−10% or +/− one or two standard deviations) for the parameter for at least 10 batches of the target protein.

131. The method of claim 115, wherein the test protein is a glycoprotein.

132. The method of claim 115, wherein the test protein is an antibody, e.g., a CDR-grafted antibody, a humanized antibody, a human antibody, or a glycosylated therapeutic antibody.

133.-134. (canceled)

135. The method of claim 115, wherein the test protein is a glycoprotein, e.g., an antibody, and said plurality of parameters comprises at least 2, 3, 4, 5, 6, 7, 8, 9, or 10 of the following parameters: HM3 glycan, HM5 glycan, Bisecting glycan A, Bisecting glycan B, C-terminal amino acid, e.g., lysine, content, sialylated glycan, a G0F glycan described herein, a G1F glycan described herein, a G2F glycan described herein, or terminal galactose-alpha-1-3-galactose.

136.-141. (canceled)

142. The method of claim 115, comprising generating a sameness/identity (s/i) value for the test protein.

143. The method of claim 115, further comprising
providing a unique seriousness value for at least one determinative test protein parameter of said plurality of determinative test protein parameters, wherein said seriousness value is a function of a risk associated with variation in the parameter associated with the determinative test protein parameter; and
generating a sameness/identity value for said test protein based on the plurality of comparisons and seriousness values.

144. The method of claim 143, wherein said seriousness value is a function of the level of terminal galactose-alpha-1-3-galactose in said test protein preparation.

145. The method of claim 143, wherein seriousness values for at least 2, 3, 4, 5, 6, 7, 8, 9, 10, or 15 parameters associated with said test protein are provided.

146. The method of claim 143, wherein said risk is a risk associated with safety or efficacy.

147. The method of claim 115 or 143, wherein said input value further comprises:
a non-determinative entry, wherein the non-determinative entry is a value for a parameter that does not distinguish said test protein from one, some or all of the non-test protein of said plurality of non-test proteins.


SEQUENCE LISTING (16 sequences)

SEQ ID NO: 1 - protein, 451 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 451 aa, Artificial Sequence
  Description of Artificial Sequence Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Description of Artificial Sequence Synthetic polypeptide
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 445 aa, Artificial Sequence
  Description of Artificial Sequence Synthetic polypeptide
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 448 aa, Artificial Sequence
  Description of Artificial Sequence Synthetic polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPSATVLMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 215 aa, Artificial Sequence
  Description of Artificial Sequence Synthetic polypeptide
EIVITQSPGTLSLSPGERATLSCRASQSVRGRYLAWYQQKPGQAPRILLYGASSRATGLPDRFSGSGSGTDFTLTISRNKPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 451 aa, Artificial Sequence
  Description of Artificial Sequence Synthetic polypeptide
EVQLVESGGGLVQPGGSLRLSCAVSGYSITSGYSWNWIRQAPGKGLEWVASITYDGSTNYNPSVKGRLTISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 218 aa, Artificial Sequence
  Description of Artificial Sequence Synthetic polypeptide
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC